(19) United States
(12) Patent Application Publication
(10) Pub. No.: US 2005/0164253 A1
(43) Pub. Date: Jul. 28, 2005

(54) Method for evaluating multiple sclerosis

(76) Inventors: Takashi Yamamura, Tokyo (JP); Junichi Satoh, Kodaira (JP); Toshiro Saito, Hatoyama (JP)

Correspondence Address: REED SMITH LLP, Suite 1400, 3110 Fairview Park Drive, Falls Church, VA, 22042, US

(21) Appl. No.: 11/000,924

(22) Filed: Dec. 2, 2004

(30) Foreign Application Priority Data
Dec. 5, 2003 (JP) 2003-406750

(51) Int. Cl. C12Q 1/68

(52) U.S. Cl. 435/6

(57) ABSTRACT

This invention provides a method and a means for assisting in the diagnosis of multiple sclerosis. More particularly, this invention provides gene markers (shown in Tables 1 and 2) for evaluating whether or not multiple sclerosis has been developed, a method for evaluating multiple sclerosis using such gene markers, a chip, and the like.

The present application claims the priority from Japanese Patent Application No. 2003-406750 filed on Dec. 5, 2003, the content of which is hereby incorporated by reference into this application.

### TECHNICAL FIELD

The present invention relates to a method of evaluation for assisting in the diagnosis of multiple sclerosis. More particularly, the present invention relates to a method for analyzing the expression of genes associated with multiple sclerosis, a chip for analyzing the expression of multiple sclerosis-associated genes, and a gene group for determining whether or not multiple sclerosis has been developed.

### BACKGROUND ART

Multiple sclerosis (hereafter abbreviated as “MS”) develops a variety of symptoms, such as visual motor sensory, and cognitive disturbances. This is because the “myelin” that covers the nerve fibers of the brain and the spinal cord become inflamed, and the transmission of neural information becomes insufficient. The cause of MS has not yet been elucidated, and MS is a chronic disease that cannot be completely cured by contemporary medicine. MS is regarded as an “autoimmune disease,” whereby the immune system erroneously attacks itself, although the mechanism of disease development has not yet been elucidated. At present, it is estimated that at least 5,000 patients with MS are present in Japan and that as many as about 1,000,000 MS patients are present in the world.

One feature of MS is that a majority of patients suffer from relapses many times. The severity and duration of relapse varies depending on the patient, and the rate of a patient recovering from MS becomes relatively high during remission after the acute stage. This type of MS is referred to as “relapsing-remitting MS.” Some patients suffer from increased neurological deficits MS as they experience repeated relapse. In contrast, there is another form of MS in which the disease conditions gradually progress after development of MS. This type of MS is referred to as “progressive MS.” The number of patients affected with the latter type is considered to be small in Japan.

MS is roughly classified in two categories in terms of the affected areas: conventional MS (C-MS) that extensively affects the entire central nervous system including the brain, the cerebellum, and the brain stem; and opticospinal MS (OS-MS) that relatively selectively affects the optic nerve and the spinal cord. While a majority of western Caucasians contract C-MS but rarely contract OS-MS, approximately one third of Asian patients with MS, including Japanese patients, contract OS-MS.

Up to the present, magnetic resonance imaging (MRI), cerebrospinal fluid (CSF) examination, and other techniques have been employed as the methods for diagnosis of MS. MRI is very useful in terms of, distinguishing active lesions from inactive lesions by the use of a contrast medium (gadolinium), although not all the lesions can be detected. In the case of OS-MS where there is no substantial development of lesions in the brain or in the cerebellum, MRI testing is particularly difficult. In addition, diagnosis needs to be made by a well-trained neuroradiologist in order to evaluate the development of the disease based on images. In the case of the cerebrospinal fluid (CSF) test, the cerebrospinal fluid that flows around the brain and the spinal cord is collected, and the quantity of lymphocytes, antibodies (the immunoglobulin G; IgG), and myelin basic protein are analyzed, thereby allowing inspection regarding the presence of an inflammatory lesion. Although this technique is useful, it inflicts a great burden on the patients, because of the necessity of sticking a needle into the back of a patient. Accordingly, it has been very difficult to determine whether or not MS has been developed in a simple accurate, and less time-consuming manner by conventional testing techniques, from the viewpoint of detection sensitivity and the burdens on test subjects.

### DISCLOSURE OF THE INVENTION

An object of the present invention is to provide a method of evaluation for assisting in the diagnosis of multiple sclerosis that provides useful information, inflicts fewer burdens on a subject, is simple, and is highly reliable.

In order to attain the above object, the present inventors have conducted concentrated studies. As a result, they have found that analysis of the expression level of a specific gene in the peripheral blood lymphocytes of the test subject enables the evaluation of whether or not MS has been developed. This has led to the completion of the present invention.

Hereafter, specific means for attaining the object are described.

The present invention relates to a method for evaluating whether or not a subject has been affected with MS by analyzing the gene expression levels of proteins associated with apoptosis inhibition or activation using messenger RNA isolated from peripheral blood lymphocytes of the subject.

The present invention also relates to a method for evaluating whether or not a subject has contracted MS by analyzing the expression level of a gene selected from among those indicated by the symbols RIPK2, NFKBIE, TNFAIP3, DAXX, TNFSF10, BAG1, TOP1, ADPRT, CREB1, MYC, BAG4, RBBP4, GZMA, BCL2, and E2F5 using messenger RNA derived from peripheral blood lymphocytes of the subject.

Also, the present invention relates to a method for evaluating whether or not a subject has contracted MS by analyzing the expression level of any of the genes, the symbols of which are shown in Table 1, using messenger RNA derived from peripheral blood lymphocytes of the subject.

Also, the present invention relates to a method for evaluating whether or not a subject has contracted MS by analyzing the expression level of any of the genes, the symbols of which are shown in Table 2, using messenger RNA derived from peripheral blood lymphocytes of the subject.

Further, the present invention relates to a method for evaluating whether or not a subject has contracted MS by isolating CD3⁺ T-cells from the peripheral blood lymphocytes of the subject and analyzing gene expression in the T-cells.

The present invention relates to a method for evaluating whether or not a subject has contracted MS wherein a DNA chip is used as a means for analyzing gene expression.

The present invention further relates to a DNA chip for evaluating whether or not MS has been developed, which has the aforementioned gene mounted thereon.

The present invention has been completed based on the results of studying the method for evaluating whether or not MS has been developed by analyzing the expression levels of a specific gene group in the peripheral blood lymphocytes of the subject via a simple means such as a DNA chip. The use of the method of evaluation according to the present invention enables the diagnosis of MS in a simple and accurate manner.

### BRIEF DESCRIPTION OF THE DRAWINGS

FIG. 1 shows the results of cluster analysis of T-cell-derived samples obtained from 66 MS patients and 17 healthy volunteers.

FIG. 2 shows the results of cluster analysis of non-T-cell-derived samples obtained from 66 MS patients and 17 healthy volunteers.

FIG. 3 shows the results of cluster analysis of T-cell-derived samples further comprising samples obtained from five test subjects.

FIG. 4 shows the results of cluster analysis of non-T-cell-derived samples further comprising samples obtained from five test subjects.

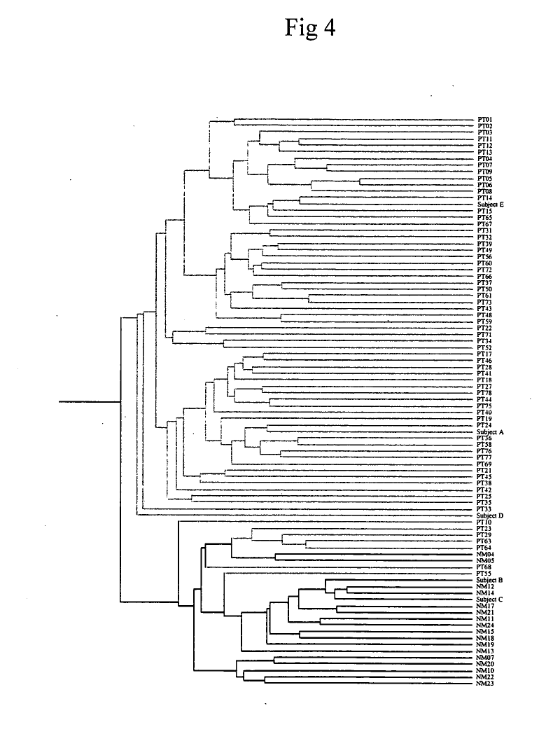

FIG. 5 shows data (1) related to the cluster analysis shown in FIG. 1.

FIG. 6 shows data (2) related to the cluster analysis shown in FIG. 1.

FIG. 7 shows data (3) related to the cluster analysis shown in FIG. 1.

FIG. 8 shows data (4) related to the cluster analysis shown in FIG. 1.

FIG. 9 shows data (5) related to the cluster analysis shown in FIG. 1.

FIG. 10 shows data (6) related to the cluster analysis shown in FIG. 1.

FIG. 11 shows data (7) related to the cluster analysis shown in FIG. 1.

FIG. 12 shows data (8) related to the cluster analysis shown in FIG. 1.

FIG. 13 shows data (9) related to the cluster analysis shown in FIG. 1.

FIG. 14 shows data (10) related to the cluster analysis shown in FIG. 1.

FIG. 15 shows data (11) related to the cluster analysis shown in FIG. 1.

FIG. 16 shows data (1) related to the cluster analysis shown in FIG. 2.

FIG. 17 shows data (2) related to the cluster analysis shown in FIG. 2.

FIG. 18 shows data (3) related to the cluster analysis shown in FIG. 2.

### BEST MODE FOR CARRYING OUT THE INVENTION

Multiple sclerosis (MS) is an autoimmune disease, and malfunction of the immune system is deduced to be the cause thereof. The immune system is an extremely complicated system in which an extensive signal transducing network exists among a variety of cells, centering on T-cells and B cells. Accordingly, it is very dangerous to judge abnormality in such immune system or the repaired state thereof simply by observing individual functions of T-cells producing various cytokines such as lymphotoxin, tumor necrosis factor (TNF), interferon γ (INFγ), or transforming growth factor β (TGFβ). Thus, the present inventors have developed a method for studying the conditions of the immune system by observing the functions of a wider range of gene groups.

Recently, a method for analyzing gene expression in a sample cell has drawn attention. In this method, a large number of DNA fragments having different sequences are independently immobilized on different sites on a substrate, the resultant is referred to as a “DNA chip” or “DNA array.” A reverse transcript of messenger RNA (fluorescence-labeled or radioisotope-labeled) that had been isolated from the target cell are sprinkled on the DNA chip or DNA array, hybridization is carried out, and the degree of hybridization of the reverse transcript to the site at which DNA fragments are immobilized relating to each sequence is determined, thereby analyzing the gene expression in the sample cell. The present inventors used this DNA chip technique to extensively determine the differences in the gene expression patterns in the peripheral blood lymphocytes of healthy volunteers and in those of MS patients.

This study was conducted to use lymphocytes responsible for the immune system obtained from peripheral blood as a sample. The use of peripheral blood lymphocytes is important from the viewpoint of the less invasive way on a subject. The 72MS patients who had been diagnosed as having relapsing-remitting MS based on comprehensive evaluation via MRI test, an evoked potential test, a cerebrospinal fluid test, and clinical findings, along with 22 healthy volunteers, were asked for their cooperation. The gene expression patterns in peripheral blood lymphocytes between MS and healthy volunteers were thoroughly compared. A DNA chip (DNA chip for analyzing drug responses, Hitachi Co., Ltd.) having approximately 1,260 types of human genes associated with cytokine, signal transmission, growth factor, oncogene, or apoptosis mounted thereon was used. After approximately 10 ml of blood was taken from the subjects, lymphocytes were separated using a density gradient centrifugation medium (Ficoll-Paque PLUS®, Amersham Biosciences), and the lymphocytes were divided into CD3⁺ T-cells and CD3⁻ non-T-cells (monocytes, B cells, and NK cells) using the AutoMACS® magnetic cell separation system (Miltenyi). Subsequently, total RNA was extracted from the separated cell fractions using the RNeasy Mini Kit (Qiagen). The yield of total RNA derived from CD3⁺ T-cells was 3 to 6 μg, and that of total RNA derived from CD3⁻ non-T-cells was 2 to 4 μg, per subject. Blood was sampled from the patients before the initiation of interferon β therapy.

Healthy volunteers (three individuals) were recruited, blood was taken, CD3⁺ T-cells and CD3⁻ non-T-cells were isolated, RNA was extracted therefrom, equivalent amounts of samples obtained from three volunteers were pooled, the resulting mixture was twice subjected to RNA amplification via in vitro transcription, and the amplified RNA was designated as a reference. This reference was used as a universal reference sample among all healthy volunteers and MS patients.

Total RNA extracted from CD3⁺ T-cells and CD3⁻ non-T-cells obtained from the healthy volunteer group and the patient group was subjected to RNA amplification via in vitro transcription. Thereafter, Cy5-labeled cDNA was synthesized via reverse transcription utilizing Cy5-dCTP. In contrast, the reference CD3+T-cell and CD3⁻ non-T-cell samples derived from healthy volunteers were independently subjected to reverse transcription using Cy3-dCTP to synthesize Cy3-labeled cDNA. The cDNA of the patients and healthy volunteers was mixed with the same amount of the reference cDNA, the resultant was applied to the DNA chip, and hybridization was carried out at 62° C. for 12 hours. After the washing, the fluorescence intensity at each spot was analyzed using a scanner (ScanArray 5000, GSI-Lumonics), and the ratio of the expression level of each gene between the samples obtained from the healthy volunteer or the patient and the reference was determined. Since the gene expression levels are expressed as a relative value to a common reference sample in this experiment utilizing DNA chips, differences in each gene expression level between the healthy volunteers and the patients can be easily determined.

The method of analysis is as follows. The data of the patient group and the healthy volunteer group were subjected to T-test. The gene group that exhibited statistically significant differences in expression levels between the aforementioned two groups even after considering individual (sample-sample) differences was selected. The T-test was carried out by the Bayes' estimation reported by A. Long et al. in combination with the T-test (Journal of Biochemistry, vol. 276, pp. 19937-19944, 2001), and the acceptable false positive value was determined to be 0.05. The results attained from CD3⁺ T-cell samples are shown in Table 1, and the results attained from CD3⁻ non-T-cell samples are shown in Table 2. The P values for expression ratio logarithmic values are shown in the tables. As the p value becomes smaller, the sample is determined to belong to a gene group that exhibits more significant differences in the expression level between healthy volunteers and MS patients, i.e., representing a MS-specific peripheral blood marker. All the p values for the groups of genes listed in Table 1 and in Table 2 are smaller than 1E-4, which are statistically significantly different. Thus, the gene group is determined to be reliable MS-specific peripheral blood gene signature.

Among the variable gene group shown in Table 1 or 2, the groups of genes indicated by the symbols RGS14, CHST2, NR4A2, MAPK1, SMARCA3, TPST2, ATP6D, TCF17, ARH1, HSPA1A, AGTRL2, and PTPN6 that have the p values of less than 1E-10 can be selected as the groups of genes exhibiting significant differences in expression levels. These are the most useful MS-specific peripheral blood markers.

The groups of genes indicated by the symbols CHST4, GHSR, COX15, IL18R1, AKAP11, CDC42, HSPA1L, RAB7L1, POLR2H, GRO2, PEMT, RPA1, and NFATC3 that have p values of less than 1E-5 in Table 1 and in Table 2 are also valuable as MS-specific peripheral blood markers.

The groups of genes indicated by the symbols ICAM1, CDC25B, IL1R2, CR2, CD3Z, MAD, CSF1, ARHGEF1, PRKDC, RASSF1, SCYA2, and ABCA1 that have p values of less than 1E-5 in Table 2 are also valuable as MS-specific peripheral blood markers.

Further, Table 1 and Table 2 contain a large number of groups of genes that are associated with apoptosis regulation and activation. The groups of apoptosis-associated genes indicated by the symbols RIPK2, NFKBIE, TNFAIP3, DAXX, TNFSF10, BAG1, TOP1, ADPRT, CREB1, MYC, BAG4, RBBP4, GZMA, BCL2, and E2F5 are identified as MS-specific peripheral blood markers.

According to the test comparing the healthy volunteer group and the MS patient group, the number of marker genes selected using the CD3⁺ T-cellsamples was approximately two times that selected using the CD3⁻ non-T-cellsamples. This indicates that T-cells are more useful for distinguishing MS from healthy subjects patients than non-T-cells.

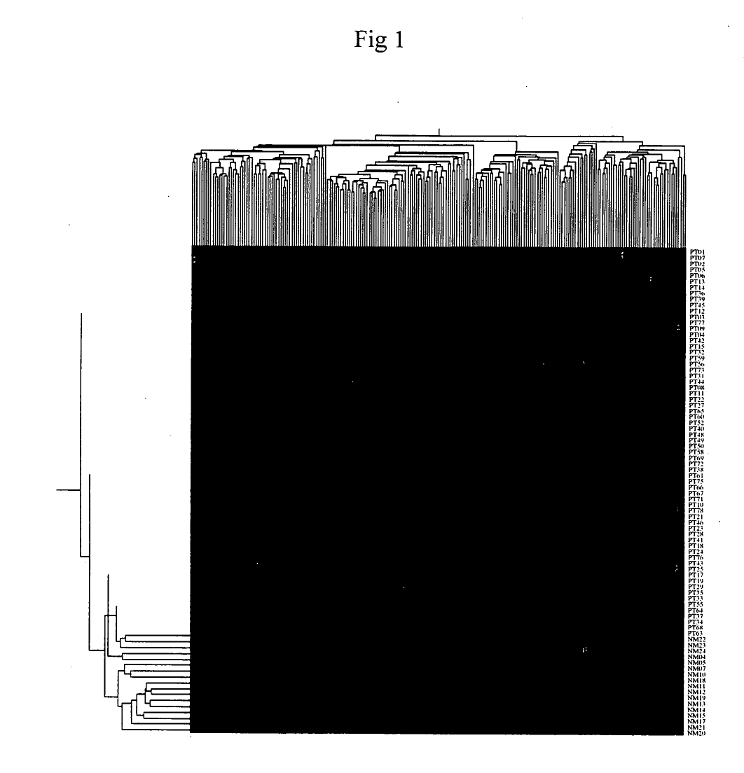

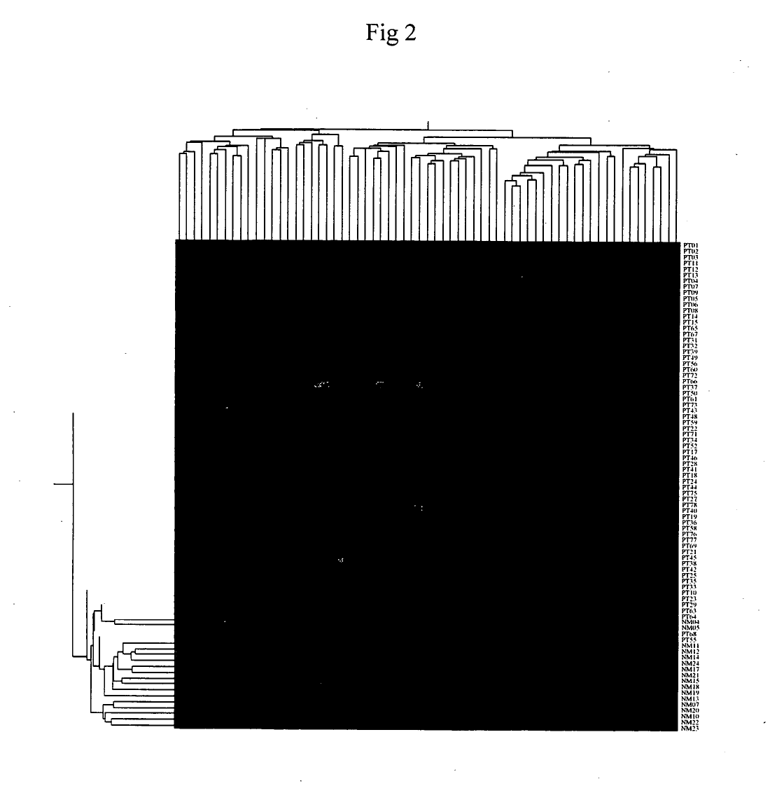

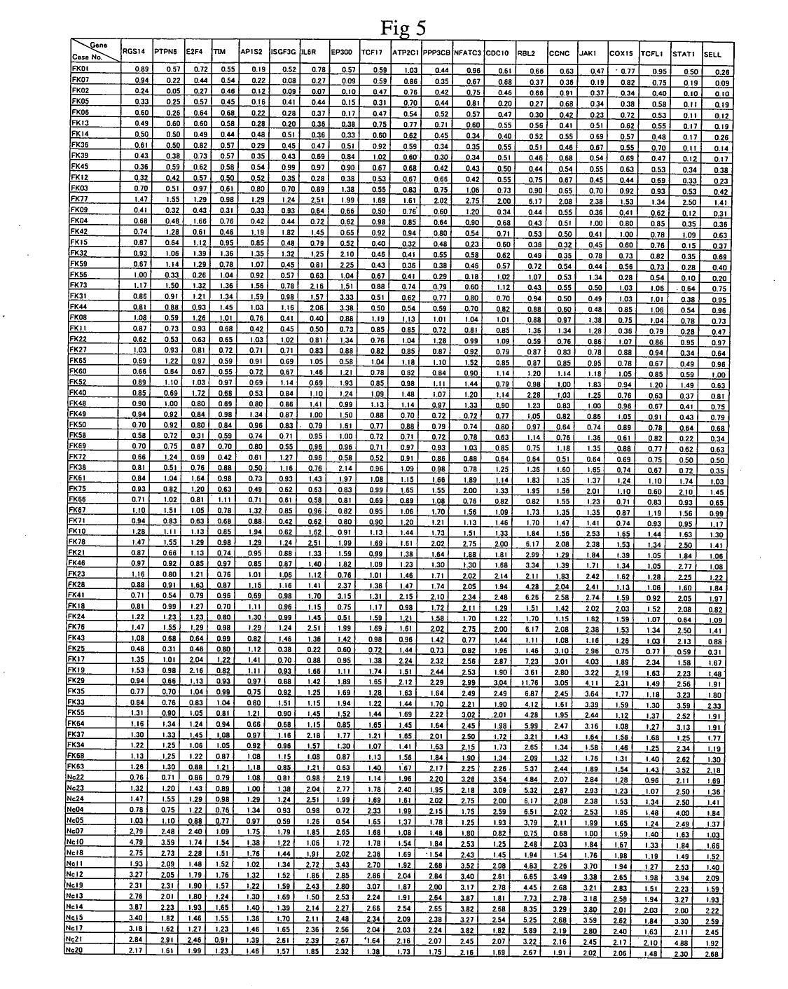

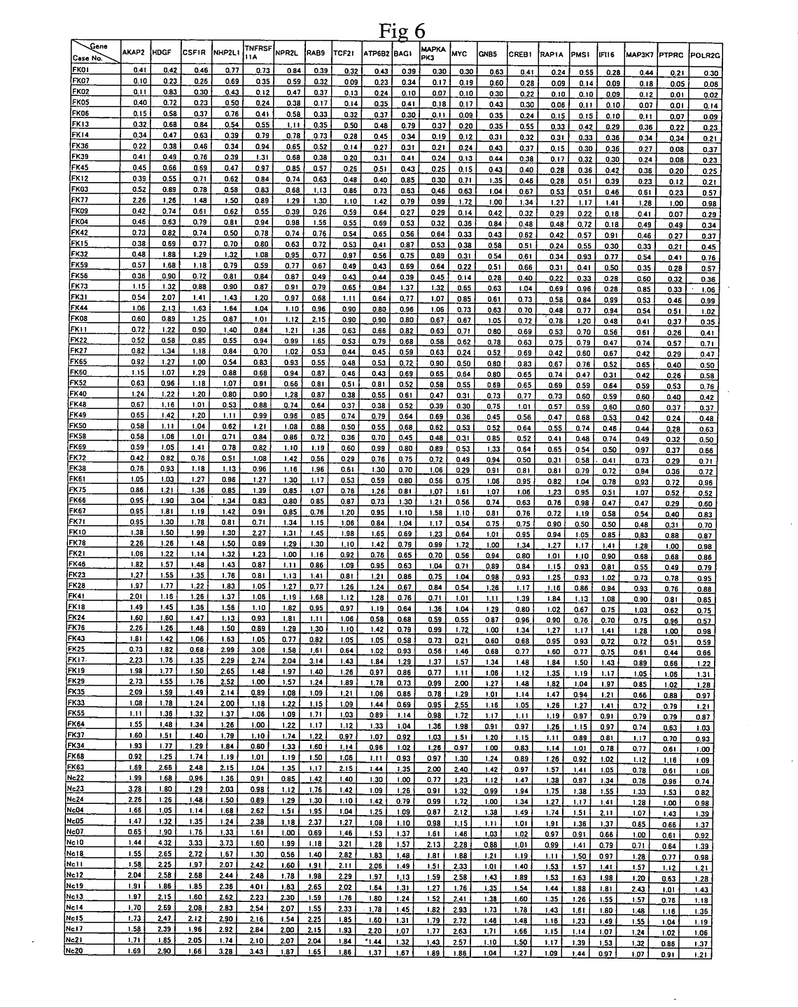

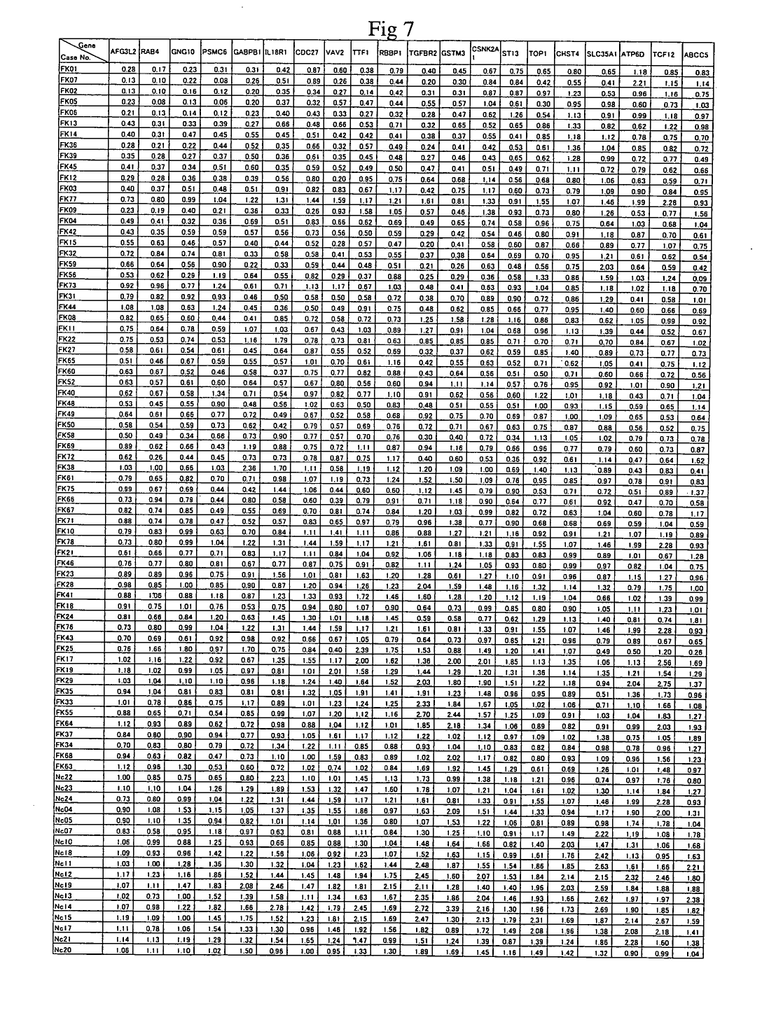

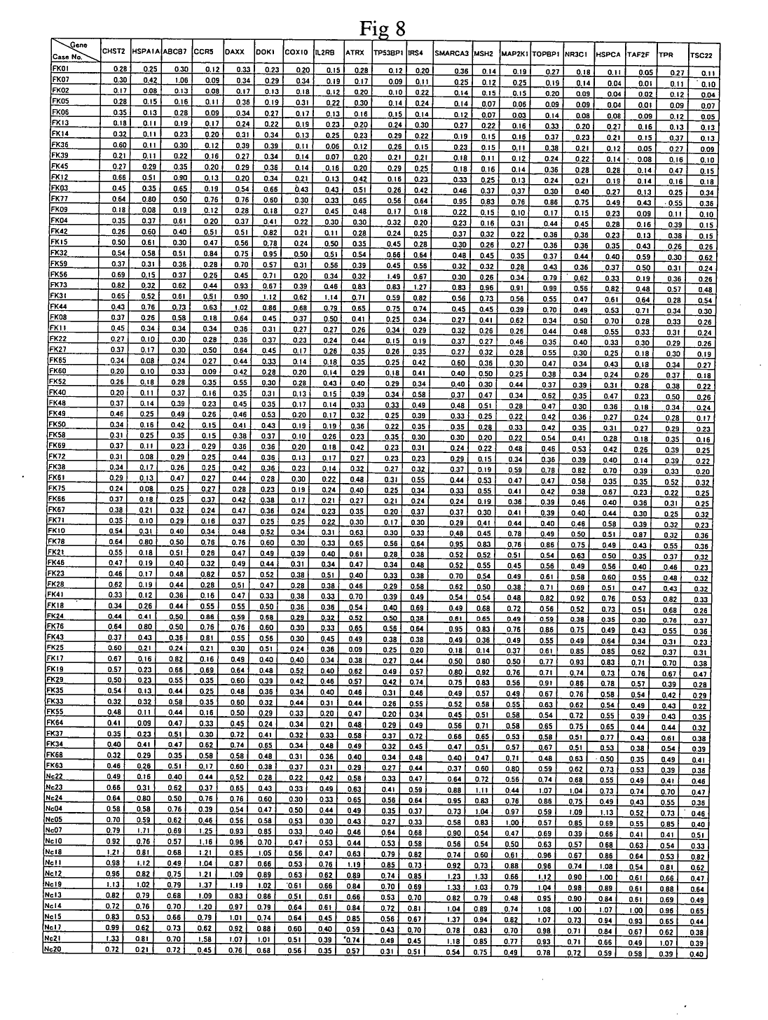

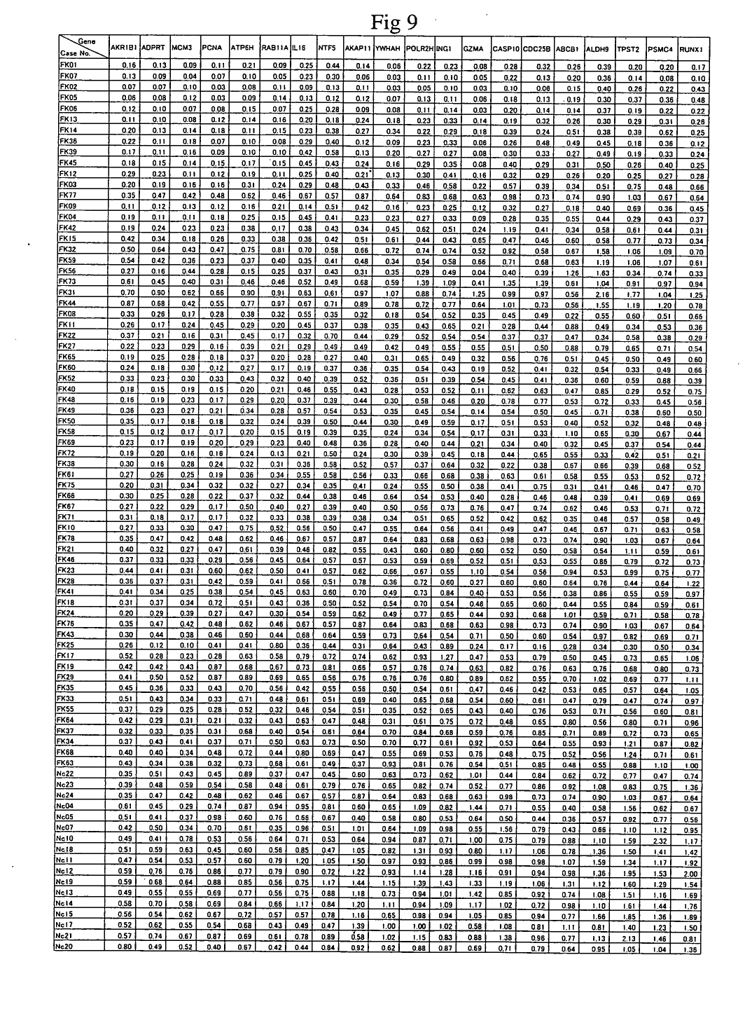

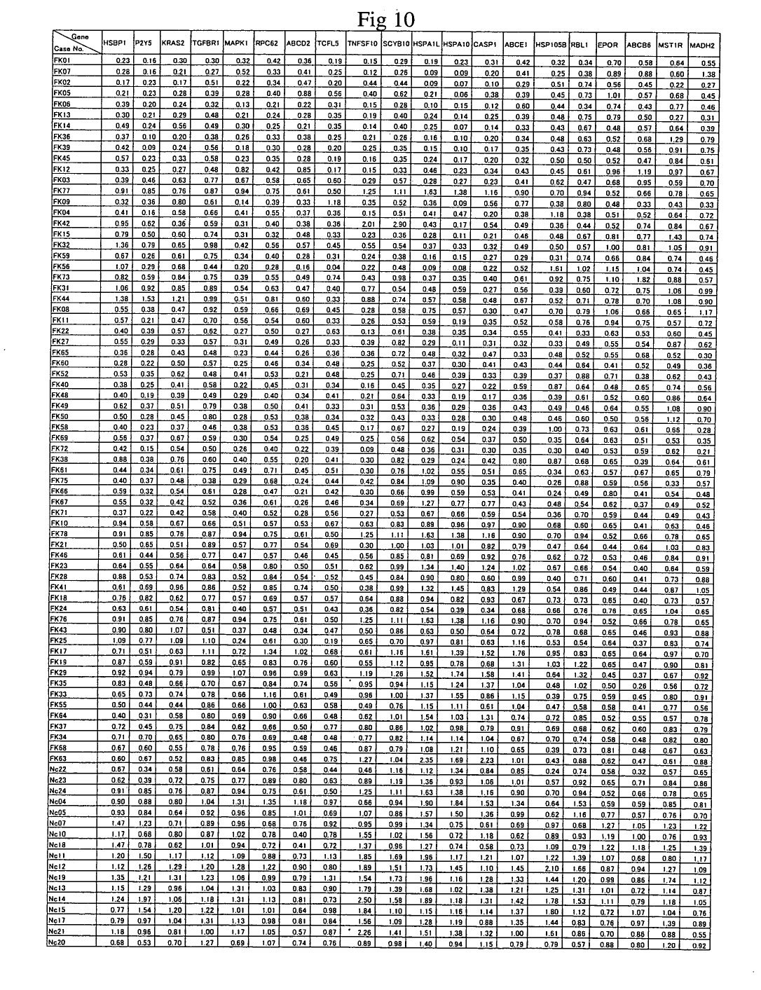

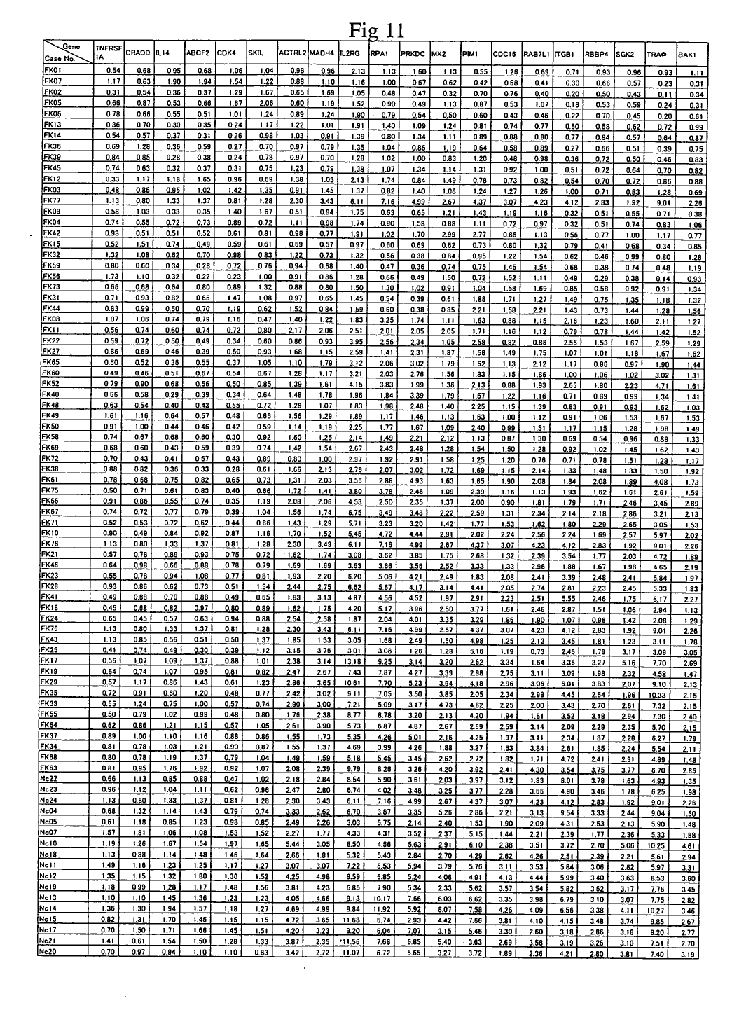

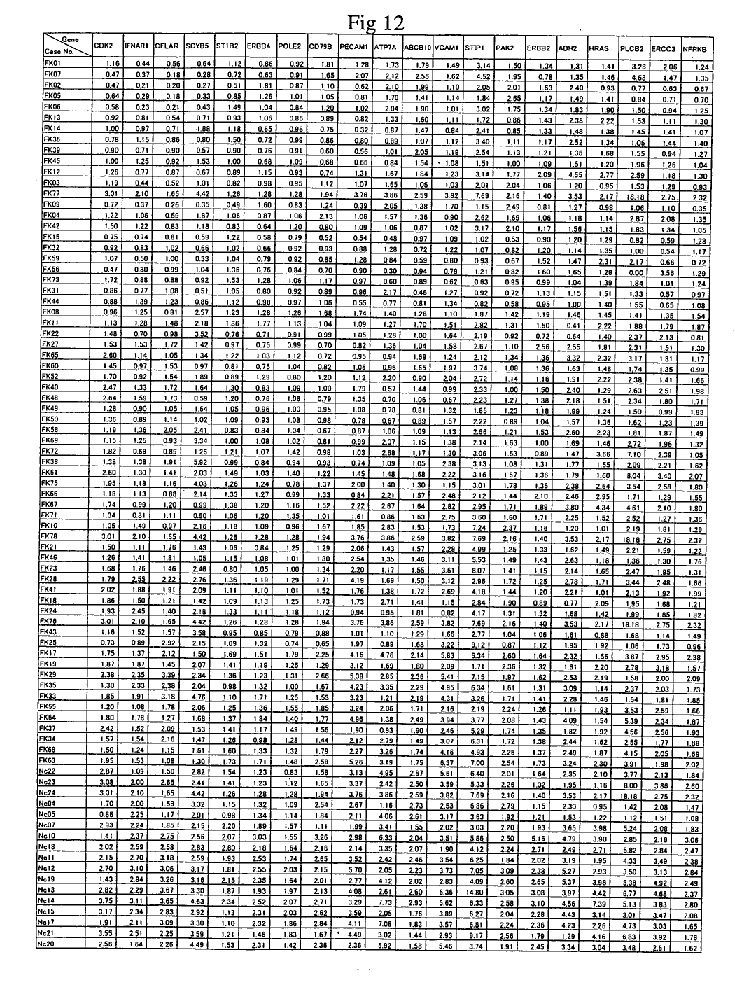

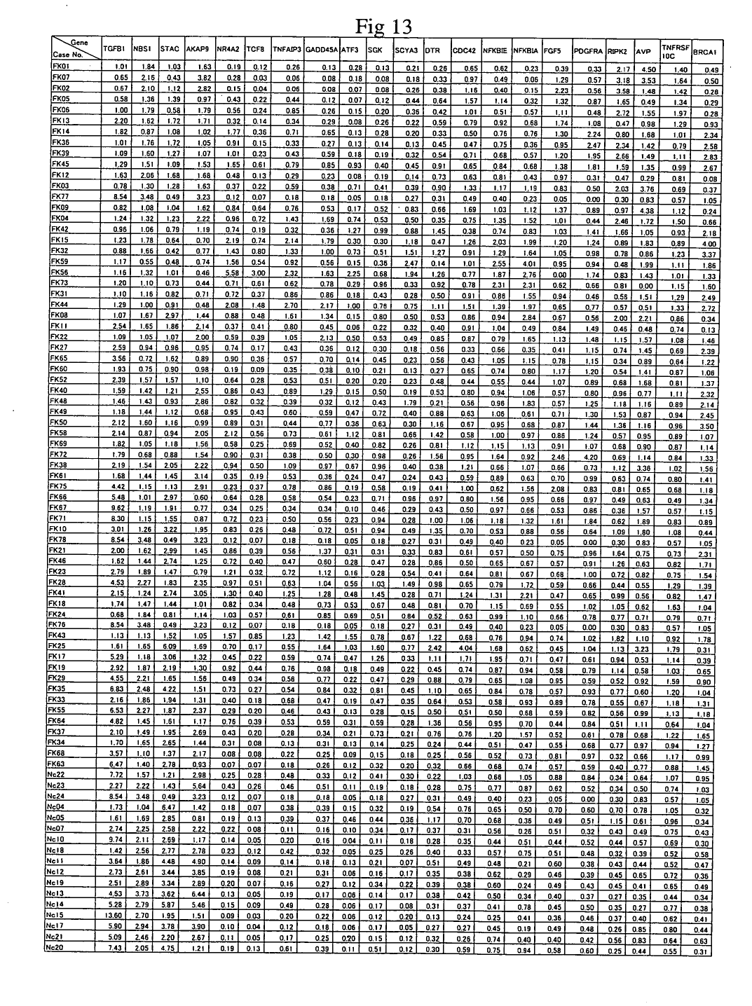

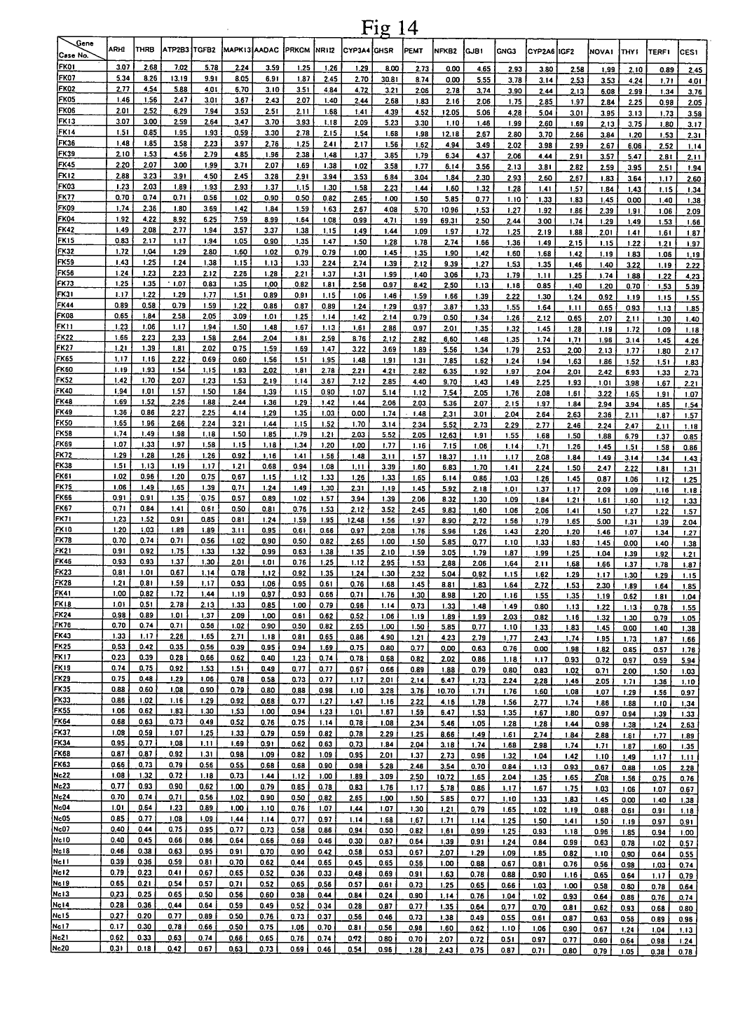

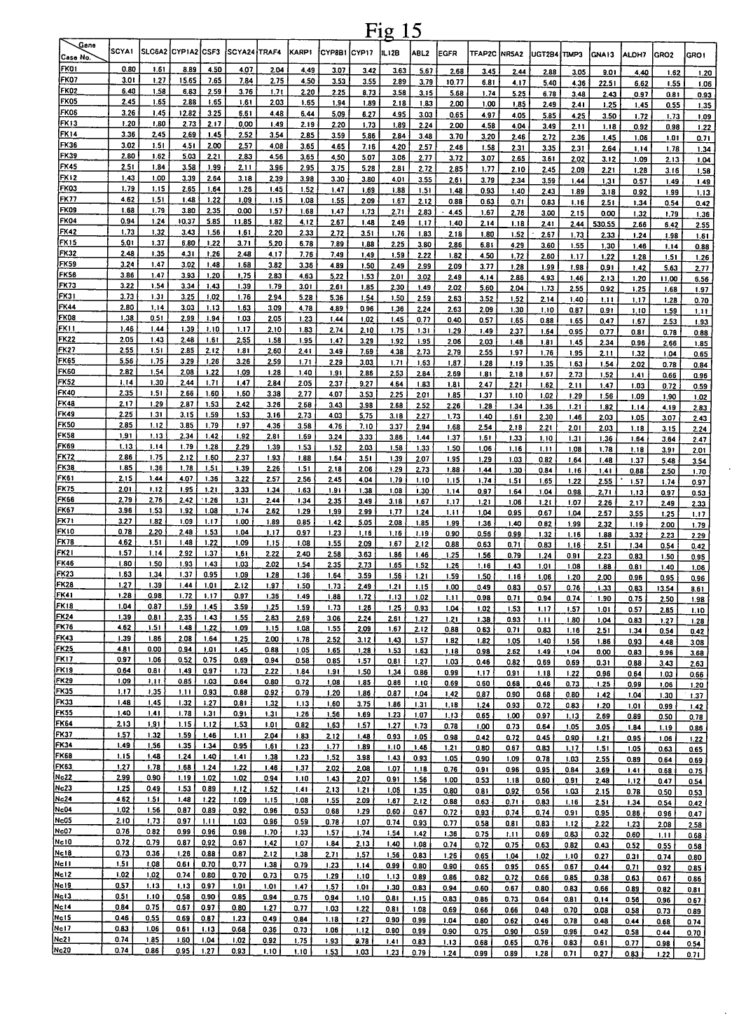

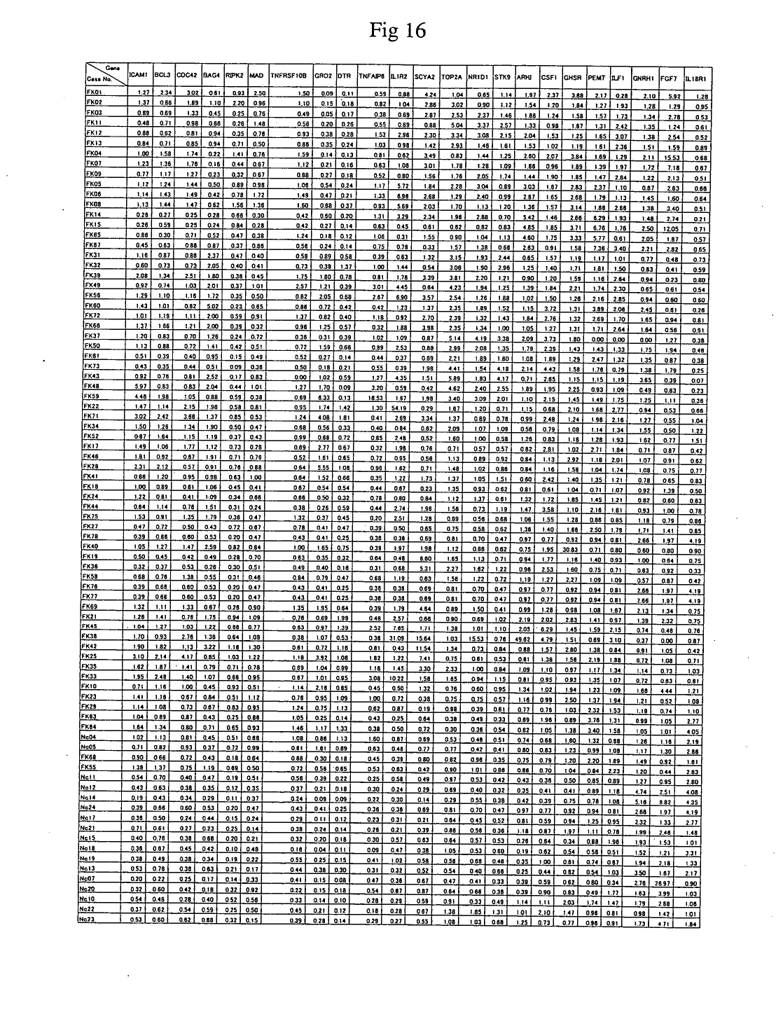

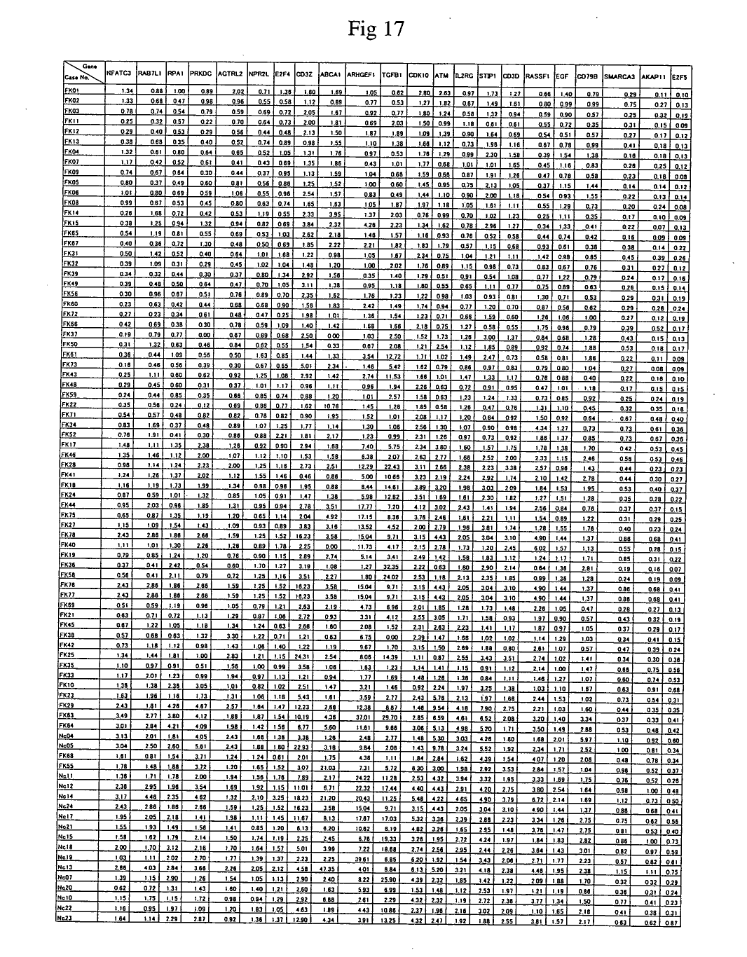

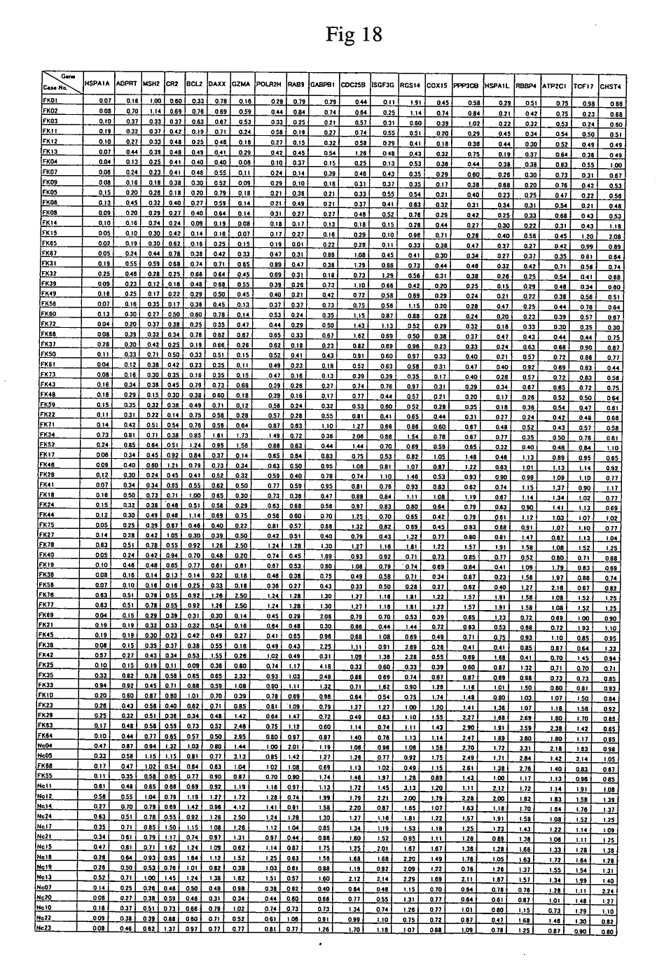

Subsequently, cluster analysis was carried out based on the expression level of the selected genes in order to group 66 MS patients and 17 healthy volunteers. The hierarchical clustering method was employed for this analysis. The resulting dendrograms are shown in FIGS. 1 and 2, the analytical data concerning FIG. 1 are shown in FIGS. 5 to 15, and the analytical data concerning FIG. 2 are shown in FIGS. 16 to 18. The vertical axis (height) is an indication of the inter-cluster distance. As is apparent from FIG. 1 and FIG. 2, the cluster of the MS patient group is clearly distinguished from that of the healthy volunteer group.

Thus, it was found that analysis of gene expression in peripheral blood lymphocytes of the subject with the use of a specific gene group as a marker enabled us to clearly distinguish of the healthy volunteer group from the patient group.

The method for analyzing the gene expression level employed in the present invention is not limited to one involving DNA chip technology. It is evident that quantitative PCR, Northern blotting, and other means can also be employed.

The method for analyzing data is not limited to one involving clustering. Machine learning algorithms, such as the Support Vector Machine, can also be employed.

The embodiments of the present invention are hereafter described in detail with reference to the examples.

### EXAMPLES

The data concerning the gene expression of the group of patients who had been clinically proved to have contracted MS and the group of healthy volunteers were stored in a database. The results of gene expression analysis of the subjects who were to be evaluated concerning the development of MS were analyzed with reference to the aforementioned database. Thus, examples of evaluation of whether or not the subjects had contracted MS were shown.

The database containing data concerning the aforementioned 66 patients and 17 healthy volunteers was employed. A total of five subjects among which three patients had been recognized as having relapsing-remitting MS based on comprehensive evaluation via MRI test, an evoked potential test, a cerebrospinal fluid test, and clinical findings and two healthy volunteers were employed. After 10 ml of peripheral blood had been taken from each subject, the origins of the samples, i.e., whether the sample was obtained from a patient or a healthy volunteer, were kept unknown via management based only on case numbers.

After lymphocytes had been separated from each blood sample using a density gradient centrifugation medium (Ficoll-Paque PLUS®, Amersham Biosciences), the lymphocytes were divided into CD3⁺ T-cells and CD3⁻ non-T-cells (monocytes, B cells, and NK cells) using the AutoMACS® magnetic cell separation system (Miltenyi). Subsequently, total RNA was extracted from the separated cell fractions using the RNeasy Mini Kit (Qiagen). The yield of total RNA derived from CD3⁺ T-cells was 3 to 6 μg, and that of total RNA derived from CD3⁻ non-T-cells was 2 to 4 μg, per subject.

At the outset, an oligo (dT) 24 primer comprising a T7 promoter sequence added thereto was annealed to 2 μg of total RNA to synthesize the first strand DNA. Subsequently, this first strand DNA was used as a template to synthesize second strand DNA having a T7 promoter sequence. Finally, the second strand DNA was used as a template to synthesize RNA with the aid of T7 RNA polymerase. A random hexamer was annealed to 4 μg of the amplified RNA to conduct reverse transcription reaction, and Cy5-dCTP was incorporated into the strand to label it with fluorescence.

The control sample was prepared in the following manner. Healthy volunteers (three individuals) were recruited, 15 ml of peripheral blood was taken from each volunteer, and CD3⁺ T-cell-derived and CD3⁻ non-T-cell-derived total RNAs were extracted, by the utilization of the aforementioned density gradient centrifugation, magnetic cell separation system, and RNA extraction kit. After 3 μg samples of total RNA obtained from each of three volunteers were pooled, Cy3-fluorescence labeled cDNA was synthesized via the aforementioned RNA amplification and reverse transcription, and the resultant was designated as the universal reference.

Cy5-cDNA prepared from each patient's sample was mixed with the same amount (4 μg) of Cy3-cDNA that was a universal reference, the mixture was applied to the aforementioned DNA chip (the DNA chip for analyzing drug responses, Hitachi Co., Ltd.), and hybridization was carried out at 62° C. for 12 hours. After washing, the fluorescence intensity at each spot was analyzed using a scanner (ScanArray® 5000, GSI-Lumonics), and quantification software (QuantArray, GSI-Lumonics) was used to determine the ratios of the gene expression intensity between the control sample and the subject sample.

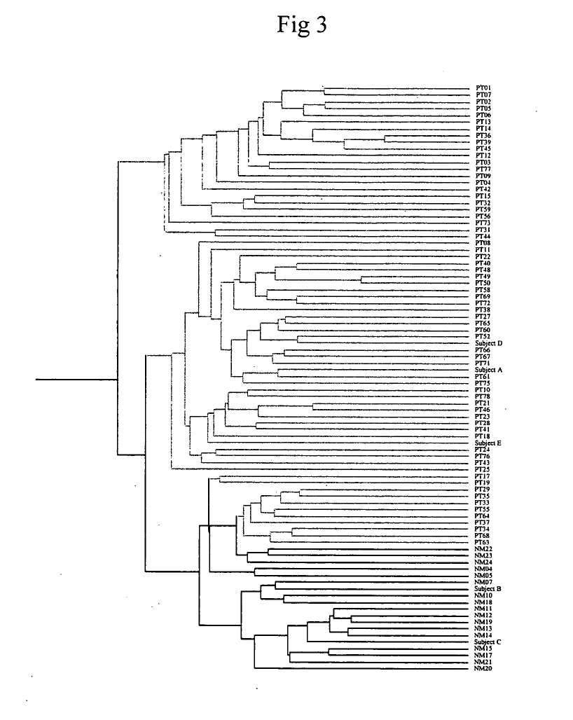

The data for these five subjects were combined with the database comprising the data concerning the aforementioned 66 patients and 17 healthy volunteers, and hierarchical clustering analysis was carried out concerning the genes shown in Table 1 and Table 2. The results attained from CD3⁺ T-cell samples utilizing the gene group shown in Table 1 are shown in FIG. 3, and the results attained from CD3⁻ non-T-cell samples utilizing the gene group shown in Table 2 are shown in Table 4. As is apparent from these figures, subjects A, D, and E among the subjects A, B, C, D, and E were classified as MS patients, and subjects B and C were classified as healthy volunteers. When the origins of the samples were traced, subjects A, D, and E were confirmed to be MS patients, and subjects B and C were confirmed to be healthy volunteers.

These results clearly indicate that analysis of the gene expression data with the utilization of the gene group shown in Table 1 and Table 2 as gene markers enables us to distinguish MS patients from healthy volunteers. This indicates that the effectiveness on diagnosis of MS by the present invention is very high. Based on the comparison of the results attained from CD3⁺ T-cells and those from CD3⁻ non-T-cells, the distinction of MS patients from healthy subjects was accurately carried out in accordance with the origins of the samples. Since CD3⁺ T-cell samples provide more accuratedistinction, the use of T-cells as peripheral blood lymphocytes was found to be the most valuable.

Among the groups of variable genes shown in Table 1 and in Table 2, the groups of genes indicated by the symbols RGS14, CHST2, NR4A2, MAPK1, SMARCA3, TPST2, ATP6D, TCF17, ARHI, HSPA1A, AGTRL2, and PTPN6 that have p values of less than 1E-10; the groups of genes indicated by the symbols CHST4, GHSR, COX15, IL18R1, AKAP11, CDC42, HSPA1L, RAB7L1, POLR2H, GRO2, PEMT, RPA1, and NFATC3 that have p values of less than 1E-5 in Table 1 and in Table 2; and the groups of genes indicated by the symbols ICAM1, CDC25B, IL1R2, CR2, CD3Z, MAD, CSF1, ARHGEF1, PRKDC, RASSF1, SCYA2, and ABCA1 that have p values of less than 1E-5 in Table 2 be considered as particularly useful gene markers for evaluating whether or not MS has been developed.

The groups of apoptosis-associated genes indicated by the symbols RIPK2, NFKBIE, TNFAIP3, DAXX, TNFSF10, BAG1, TOP1, ADPRT, CREB1, MYC, BAG4, RBBP4, GZMA, BCL2, and E2F5 are also particularly useful as gene markers for evaluating whether or not MS has been developed.

The groups of genes shown in Table 1 are useful as gene markers for evaluating whether or not MS has been developed in the case of T-cell-derived samples. The groups of genes shown in Table 2 are useful as gene markers for the aforementioned purpose in the case of non-T-cell-derived samples.

The thus selected groups of genes can be employed for diagnosing MS if a chip having a probe that specifically binds to the gene group immobilized on the surface thereof is prepared for at least some of those genes.

TABLE 1
List of genes exhibiting variable expression in T-cell-derived samples

| | | | GenBank | |
|---|---|---|---|---|
| Symbol | Name | Category | (Acc. No.) | p-log |
| RGS14 | Homo sapiens regulator of G protein | Signal | AF037195 | 1.51E−13 |
| | signaling RGS14 mRNA, complete cds. | | | |
| CHST2 | Homo sapiens carbohydrate | sulfotransferase | NM_004267 | 6.43E−13 |
| | (N-acetylglucosamine-6-O) | | | |
| | sulfotransferase 2 (CHST2) | | | |
| NR4A2 | H. sapiens mRNA for NOT | NR4, TF | X75918 | 2.55E−12 |
| MAPK1 | Human extracellular signal-regulated | Signal | M84489 | 6.02E−12 |
| | kinase 2 mRNA; ERK2 | | | |
| SMARCA3 | SWI/SNF related, matrix associated, actin | ATPase | Z46606 | 1.70E−11 |
| | dependent regulator of chromatin, | | | |
| | subfamily a, member 3 | | | |
| TPST2 | Homo sapiens tyrosylprotein | sulfotransferase | AF049891 | 2.31E−11 |
| | sulfotransferase-2 mRNA | | | |
| ATP6D | ATPase, H+ transporting, lysosomal | ATPase | J05682 | 2.46E−11 |
| | (vacuolar proton pump) 42 kD; Vacuolar | | | |
| | proton-ATPase, subunit C; V-ATPase, | | | |
| | subunit C | | | |
| TCF17 | Homo sapiens HKL1 mRNA, complete cds | Signal, TF | D89928 | 3.14E−11 |
| ARHI | Homo sapiens putative tumor supressor | Signal, suppressor | U96750 | 3.82E−11 |
| | NOEY2 mRNA; Ras homolog gene family, | | | |
| | member I | | | |
| HSPA1A | Homo sapiens heat shock 70 kD protein 1 | hsp | NM_005345 | 4.67E−11 |
| | (HSPA1A), mRNA; Heat shock 70 kD | | | |
| | protein 1 | | | |
| AGTRL2 | Homo sapiens angiotensin receptor-like 2 | angiotensin | NM_005162 | 3.51E−10 |
| | (AGTRL2) | | | |
| TNFSF10 | Human TNF-related apoptosis inducing | Cytokine | U37518 | 5.19E−10 |
| | ligand TRAIL mRNA, complete cds | | | |
| TOP1 | Human topoisomerase I mRNA, complete cds | topoiosomerase | J03250 | 7.03E−10 |
| PTPN6 | H. sapiens PTP1C mRNA for | Signal | X62055 | 7.77E−10 |
| | protein-tyrosine phosphatase 1C.; Protein | | | |
| | tyrosine phosphatase, non-receptor type 6; SHP-1 | | | |
| CCR5 | Human CC chemokine receptor 5 (CCR5) | Signal | U54994 | 1.10E−09 |
| | mRNA, complete cds | | | |
| TCF8 | Human mRNA for transcription factor | Cytokine, Signal, TF | D15050 | 1.17E−09 |
| | AREB6; Transcription factor 8 (represses | | | |
| | interleukin 2 expression) | | | |
| CHST4 | Homo sapiens carbohydrate | sulfotransferase | NM_005769 | 1.84E−09 |
| | (N-acetylglucosamine 6-O) | | | |
| | sulfotransferase 4 (CHST4) | | | |
| ERBB4 | Homo sapiens receptor tyrosine kinase | oncogene | L07868 | 2.22E−09 |
| | (ERBB4) gene, complete cds | | | |
| GHSR | Homo sapiens growth hormone | GH | NM_004122 | 4.60E−09 |
| | secretagogue receptor (GHSR) | | | |
| TCF21 | Homo sapiens epicardin mRNA, complete cds. | Signal, TF | AF047419 | 4.99E−09 |
| ATP6B2 | ATPase, H+ transporting, lysosomal | ATPase | L35249 | 5.10E−09 |
| | (vacuolar proton pump), beta polypeptide, | | | |
| | 56/58 kD, isoform 2 | | | |
| CREB1 | Homo sapiens cAMP responsive element | ATF/CREB | NM_004379 | 6.58E−09 |
| | binding protein 1 (CREB1) | | | |
| ITGB1 | Integrin, beta 1 (fibronectin receptor, beta | Signal | X07979 | 7.16E−09 |
| | polypeptide, antigen CD29 includes MDF2, MSK12); | | | |
| THRB | Human c-erb-A mRNA for thyroid hormone | oncogene | X04707 | 9.10E−09 |
| | receptor | | | |
| COX15 | Homo sapiens COX15 (yeast) homolog, | mitochondria & stress | NM_004376 | 1.13E−08 |
| | cytochrome c oxidase assembly protein (COX15) | | | |
| MYC | Human mRNA encoding the c-myc oncogene | oncogene, Signal, TF | V00568 | 1.18E−08 |
| BAG1 | Homo sapiens Bcl-2-binding protein | glucocorticoids | AF022224 | 1.51E−08 |
| | (BAG-1) mRNA | (Cortisol) | | |
| CYP1A2 | Homo sapiens cytochrome P450, subfamily | P450 | NM_000761 | 1.64E−08 |
| | I (aromatic compound-inducible), | | | |
| | polypeptide 2 (CYP1A2) mRNA | | | |
| CDC16 | Human CDC16Hs mRNA, complete cds | CellCycle | U18291 | 1.99E−08 |
| SLC35A1 | solute carrier family 35 (CMP-sialic acid | polymerase | D87969 | 2.06E−08 |
| | transporter), member 1 | | | |
| DAXX | Homo sapiens Fas-binding protein Daxx | Signal | AF015956 | 2.23E−08 |
| | mRNA, complete cds | | | |
| TSC22 | Human putative regulatory protein | GF | U35048 | 2.34E−08 |
| | TGF-beta-stimulated clone 22 homolog (TSC22) | | | |
| GABPB1 | Homo sapiens GA-binding protein | mitochondria & stress | NM_005254 | 6.16E−08 |
| | transcription factor, beta subunit 1 (53 kD); | | | |
| | nuclear respiratory factor-2 | | | |
| ADPRT | Human poly(ADP-ribose) polymerase | Signal | M18112 | 6.72E−08 |
| | mRNA (ADPRT), PARP | | | |
| MCM3 | minichromosome maintenance deficient (S. | polymerase | D38073 | 6.97E−08 |
| | cerevisiae) 3 | | | |
| IL14 | Homo sapiens clone 24607 mRNA | Cytokine | AF070546 | 7.69E−08 |
| | sequence | | | |
| IL18R1 | Human putative transmembrane receptor | Cytokine, Signal | U43672 | 8.57E−08 |
| | IL-1Rrp mRNA, complete cds | | | |
| ATP2B3 | ATPase, Ca++ transporting, plasma | ATPase | U57971 | 8.64E−08 |
| | membrane 3 | | | |
| GJB1 | gap junction protein, beta 1, 32 kD | Gap-junciton | X04325 | 1.03E−07 |
| | (connexin 32, Charcot-Marie-Tooth | | | |
| | neuropathy, X-linked) | | | |
| PIM1 | Human h-pim-1 protein (h-pim-1) mRNA, | oncogene | M54915 | 1.20E−07 |
| | complete cds | | | |
| CYP2A6 | Human cytochrome P450IIA3 (CYP2A3) | P450 | M33318 | 1.28E−07 |
| | mRNA, complete cds | | | |
| CES1 | Human carboxylesterase mRNA | esterase | L07765 | 1.36E−07 |
| NR1I2 | Homo sapiens orphan nuclear receptor PXR | NR1(PXR) | AF061056 | 1.43E−07 |
| | mRNA, complete cds | | | |
| AKAP11 | A kinase (PRKA) anchor protein 11 | Signal | AB014529 | 1.56E−07 |
| | (AKAP11); Homo sapiens mRNA for | | | |
| | KIAA0629 protein, partial cds | | | |
| CD79B | Human immunoglobulin superfamily | Signal | M89957 | 1.64E−07 |
| | member B cell receptor complex cell | | | |
| | surface glycoprotein (IGB) mRNA, CD79B | | | |
| MSH2 | Human DNA mismatch repair protein MSH2 | DNArepair | U04045 | 1.82E−07 |
| CDC42 | Human GTP-binding protein (G25K) | CellCycle | M35543 | 1.91E−07 |
| | mRNA, complete cds | | | |
| MAP3K7 | Homo sapiens mitogen-activated protein | Signal | NM_003188 | 2.22E−07 |
| | kinase kinase kinase 7 (MAP3K7), mRNA, TAK1 | | | |
| RBBP4 | Human chromatin assembly factor 1 p48 | Signal | X74262 | 2.36E−07 |
| | subunit (CAF1 p48 subunit); | | | |
| | retinoblastoma-binding protein 4 | | | |
| GNA13 | Human guanine nucleotide regulatory | Signal | L22075 | 2.45E−07 |
| | protein (G13) mRNA; Guanine nucleotide | | | |
| | binding protein (G protein), alpha 13 | | | |
| TCF12 | Homo sapiens transcription factor (HTF4) | Signal, TF | M83233 | 2.48E−07 |
| | mRNA, complete cds | | | |
| TIM | Human guanine nucleotide regulatory | oncogene | U02082 | 2.54E−07 |
| | protein (tim1) mRNA, complete cds. | | | |
| TNFAIP3 | Human tumor necrosis factor alpha | Cytokine, Signal | M59465 | 2.59E−07 |
| | inducible protein A20 mRNA complete cds | | | |
| HSPA1L | Homo sapiens HSPA1L mRNA for Heat | hsp | D85730 | 3.18E−07 |
| | shock protein 70 testis variant, complete | | | |
| | cds; Heat shock 70 kD protein-like 1 | | | |
| TCFL5 | Homo sapiens TCFL5 mRNA for | Signal, TF | AB012124 | 3.28E−07 |
| | transcription factor-like 5, complete cds | | | |
| RAB7L1 | Homo sapiens mRNA for small | oncogene | D84488 | 3.86E−07 |
| | GTP-binding protein, complete cds | | | |
| POLR2H | Human RNA polymerase II subunit | polymerase | U37689 | 4.21E−07 |
| | (hsRPB8) mRNA; polymerase (RNA) II | | | |
| | (DNA directed) polypeptide H | | | |
| ATP2C1 | ATPase, Ca++-sequestering | ATPase | AF225981 | 4.28E−07 |
| ATP7A | ATPase, Cu++ transporting, alpha | ATPase | L06133 | 5.01E−07 |
| | polypeptide (Menkes syndrome) | | | |
| RIPK2 | Homo sapiens serine/threonine kinase | Appoptosis, Signal | AF027706 | 5.31E−07 |
| | RICK (RICK) mRNA; RIP2 | | | |
| NFKBIE | Human I kappa B epsilon (IkBe) mRNA, | Signal | U91616 | 5.72E−07 |
| | complete cds | | | |
| TNFRSF11A | Homo sapiens receptor activator of nuclear | Cytokine | AF018253 | 6.14E−07 |
| | factor-kappa B (RANK) mRNA, complete cds | | | |
| ERBB2 | Human tyrosine kinase-type receptor | oncogene | M11730 | 6.16E−07 |
| | (HER2) mRNA; ERBB2; neu proto-oncogene | | | |
| CASP10 | Human apoptotic cysteine protease Mch4 | Appoptosis, Signal | U60519 | 6.86E−07 |
| | (Mch4) mRNA, complete cds | | | |
| GZMA | Human Hanukah factor serine protease | esterase | M18737 | 6.89E−07 |
| | (HuHF) mRNA (cytotoxic | | | |
| | T-lymphocyte-associated serine esterase 3) | | | |
| PSMC4 | Proteasome (prosome, macropain) 26S | ATPase | AF020736 | 7.18E−07 |
| | subunit, ATPase, 4 | | | |
| IFNAR1 | Human interferon-alpha receptor | Cytokine, Signal | J03171 | 7.39E−07 |
| | (HuIFN-alpha-Rec) mRNA, complete cds | | | |
| TRAF4 | H. sapiens MLN62 mRNA (TNF | Cytokine | X80200 | 7.44E−07 |
| | receptor-associated factor 4) | | | |
| NOVA1 | Human onconeural ventral antigen-1 | oncogene | U04840 | 7.84E−07 |
| | (Nova-1) mRNA, complete cds | | | |
| ABCF2 | Homo sapiens clone 203 ABC transporter | ABC transporter | AF091073 | 8.15E−07 |
| | mRNA, complete cds | | | |
| DOK1 | Docking protein 1, 62 kD (downstream of | Gap-junciton | U70987 | 8.73E−07 |
| | tyrosine kinase 1) | | | |
| HSBP1 | Homo sapiens heat shock factor binding | hsp | AF068754 | 8.73E−07 |
| | protein 1 HSBP1 mRNA; Heat shock factor | | | |
| | binding protein 1 | | | |
| GRO2 | Human mRNA for macrophage | Cytokine | X53799 | 1.07E−06 |
| | inflammatory protein-2alpha (MIP2alpha,; | | | |
| | GRO2 oncogene | | | |
| PEMT | Homo sapiens mRNA for | methytransferase | AB029821 | 1.11E−06 |
| | phosphatidylethanolamine | | | |
| | N-methyltransferase, complete cds | | | |
| RUNX1 | Human AML1 mRNA for AML1b protein | oncogene | D43968 | 1.13E−06 |
| | (alternatively spliced product), complete cds | | | |
| VAV2 | VAV2 = VAV oncogene homolog [human, | oncogene | S76992 | 1.14E−06 |
| | fetal brain, mRNA Partial, 2753 bp | | | |
| ATF3 | Human activating transcription factor 3 | ATF/CREB | L19871 | 1.21E−06 |
| | (ATF3) mRNA | | | |
| P2Y5 | Homo sapiens purinergic receptor P2Y5 mRNA | Signal | AF000546 | 1.23E−06 |
| HDGF | Human mRNA for hepatoma-derived | GF | D16431 | 1.38E−06 |
| | growth factor, complete cds | | | |
| PCNA | Homo sapiens proliferating cell nuclear | CellCycle, Signal | NM_002592 | 1.42E−06 |
| | antigen (PCNA) mRNA | | | |
| NBS1 | Nijmegen breakage syndrome 1 (nibrin) | Signal | AF058696 | 1.45E−06 |
| TFAP2C | Human transcription factor ERF-1 mRNA; | TF | U85658 | 1.49E−06 |
| | Transcription factor AP-2 gamma | | | |
| | (activating enhancer-binding protein 2 gamma) | | | |
| MAPKAPK3 | Homo sapiens mitogen-activated protein | Signal | NM_004635 | 1.50E−06 |
| | kinase-activated protein kinase 3 | | | |
| TOPBP1 | Homo sapiens mRNA for DNA topoisomerase | topoiosomerase | AB019397 | 1.60E−06 |
| | II binding protein, complete cds | | | |
| AVP | Human vasopressin mRNA; Arginine | vasopressin | M25647 | 1.61E−06 |
| | vasopressin (neurophysin II, antidiuretic | | | |
| | hormone, diabetes insipidus, neurohypophyseal) | | | |
| HSP105B | Molecular cloning, expression and | hsp | AB003333 | 1.77E−06 |
| | localization of human 105 kDa heat shock | | | |
| | protein, hsp105D | | | |
| IL2RG | Human mRNA for interleukin 2 receptor | Cytokine, Signal | D11086 | 1.90E−06 |
| | gamma chain | | | |
| CYP17 | Human cytochrome P450c17 (steroid | glucocorticoids | M14564 | 1.93E−06 |
| | 17-alpha-hydroxylase/17,20 lyase) mRNA, | (Cortisol) | | |
| | complete cds. | | | |
| IL16 | Homo sapiens putative IL-16 protein | Cytokine | M90391 | 2.03E−06 |
| | precursor, mRNA, complete cds | | | |
| ST1B2 | Homo sapiens mRNA for ST1B2 | sulfotransferase | D89479 | 2.11E−06 |
| E2F4 | Homo sapiens E2F transcription factor 4, | TF | NM_001950 | 2.13E−06 |
| | p107/p130-binding (E2F4) | | | |
| YWHAH | Human 14-3-3n protein mRNA; Tyrosine | Tyrosine Hydroxylase | L20422 | 2.23E−06 |
| | 3-monooxygenase/tryptophan 5-monooxygenase | | | |
| | activation protein, eta polypeptide | | | |
| COX10 | Homo sapiens COX10 (yeast) homolog, | mitochondria & stress | NM_001303 | 2.28E−06 |
| | cytochrome c oxidase assembly protein | | | |
| | (heme A: farnesyltransferase) | | | |
| SCYB10 | Human mRNA for gamma-interferon | Cytokine | X02530 | 2.60E−06 |
| | inducible early response gene (with | | | |
| | homology to platelet proteins). | | | |
| TGFBR2 | Homo sapiens mRNA for TGF-betaIIR | GF, Signal | D50683 | 2.82E−06 |
| | alpha, complete cds | | | |
| PMS1 | Human DNA mismatch repair protein | DNA repair | U13695 | 2.90E−06 |
| | PMS1 (PMS1 protein homolog 1) | | | |
| FGF5 | Human fibroblast growth factor-5 (FGF-5) | GF | M37825 | 3.03E−06 |
| | mRNA, complete cds | | | |
| PSMC6 | Proteasome (prosome, macropain) 26S | ATPase | AF006305 | 3.06E−06 |
| | subunit, ATPase, 6 | | | |
| CDC10 | hCDC10 = CDC10 homolog [human, fetal | CellCycle | S72008 | 3.08E−06 |
| | lung, mRNA, 2314 nt]. | | | |
| RPA1 | Replication protein A1 (70 kD) | Signal | M63488 | 3.15E−06 |
| BAK1 | Human bc12 homologous antagonist/killer (BAK) | Appoptosis | U23765 | 3.25E−06 |
| PPP3CB | Human calcineurin A2 mRNA; | Signal | M29551 | 3.73E−06 |
| PECAM1 | Platelet/endothelial cell adhesion molecule | Signal | M28526 | 3.77E−06 |
| | (CD31 antigen), neutrophil; CD31 | | | |
| NFKBIA | Homo sapiens MAD-3 mRNA encoding | Signal | M69043 | 3.80E−06 |
| | IkB-like activity, complete cds, IkBalpha | | | |
| NFATC3 | Homo sapiens NF-AT4c mRNA, complete cds | Signal, TF | L41067 | 4.10E−06 |
| EPOR | Human erythropoietin receptor mRNA, complete cds | Cytokine, Signal | M60459 | 4.39E−06 |
| GADD45A | Human growth arrest and DNA-damage- | DNA-damage-inducible | M60974 | 4.55E−06 |
| | inducible protein (gadd45) mRNA | | | |
| TCFL1 | Human YL-1 mRNA for YL-1 protein (nuclear | Signal, TF | D43642 | 4.86E−06 |
| | protein with DNA-binding ability), complete cds | | | |
| TP53BP1 | Human clone 53BP1 p53-binding protein | Supressor | U09477 | 5.32E−06 |
| | mRNA, partial cds. | | | |
| IFI16 | Homo sapiens interferon, gamma-inducible | Cytokine | NM_005531 | 5.40E−06 |
| | protein 16 (IFI16) mRNA | | | |
| IL12B | Human natural killer cell stimulatory factor | Cytokine, Signal | M65290 | 5.48E−06 |
| | (NKSF) mRNA, complete cds, clone p40 | | | |
| SCYA24 | Human myeloid progenitor inhibitory | Cytokine | U85768 | 5.53E−06 |
| | factor-1 MPIF-2 mRNA | | | |
| POLE2 | polymerase (DNA directed), epsilon 2 | polymerase | AF025840 | 5.63E−06 |
| ATRX | Alpha thalassemia/mental retardation | ATPase | U72938 | 6.06E−06 |
| | syndrome X-linked | | | |
| CRADD | Human death domain containing protein | Appoptosis, Signal | U84388 | 6.12E−06 |
| | CRADD mRNA; CASP2 and RIPK1 domain | | | |
| | containing adaptor with death domain | | | |
| GRO1 | Human mRNA for melanoma growth | Signal, TF | X12510 | 6.55E−06 |
| | stimulatory activity (MGSA), groucho | | | |
| GNB5 | Homo sapiens G protein beta 5 subunit | Signal | AF017656 | 6.78E−06 |
| | mRNA; Guanine nucleotide binding protein | | | |
| | (G protein), beta 5 | | | |
| SGK2 | Homo sapiens serum/glucocorticoid | hyperosmotic stress | NM_016276 | 6.96E−06 |
| | regulated kinase 2 | | | |
| NFKB2 | H. sapiens mRNA for NF-kB subunit (p49/p100) | Signal | X61498 | 7.06E−06 |
| IRS4 | Homo sapiens insulin receptor substrate 4 | Insulin | NM_003604 | 7.17E−06 |
| | (IRS4) | | | |
| SLC6A2 | Homo sapiens solute carrier family 6 | norepinephrine | NM_001043 | 7.61E−06 |
| | (neurotransmitter transporter, | | | |
| | noradrenalin), member 2 (SLC6A2) | | | |
| RBL1 | Human retinoblastoma related protein | CellCycle | L14812 | 8.03E−06 |
| | (p107) mRNA; Retinoblastoma-like 1 | | | |
| CASP1 | Human interleukin 1-beta converting | Appoptosis, Signal | U13699 | 8.23E−06 |
| | enzyme isoform delta (IL1BCE) mRNA, complete cds | | | |
| KARP1 | Ku86 autoantigen related protein 1 | Signal | AF039597 | 8.49E−06 |
| NHP2L1 | Non-histone chromosome protein 2 (S. | Signal | D50420 | 8.50E−06 |
| | cerevisiae)-like 1 | | | |
| SGK | Homo sapiens serum/glucocorticoid | hyperosmotic stress | NM_005627 | 8.54E−06 |
| | regulated kinase | | | |
| PLCB2 | Homo sapiens phospholipase C-beta-2 | Signal | M95678 | 8.56E−06 |
| | mRNA; Phospholipase C, beta 2 | | | |
| CDK4 | Human (clone PSK-J3) cyclin-dependent | CellCycle, Signal | M14505 | 8.84E−06 |
| | protein kinase mRNA; cyclin-dependent | | | |
| | kinase 4 (CDK4) | | | |
| PRKCM | H. sapiens mRNA for protein kinase C mu; | Signal | X75756 | 8.93E−06 |
| | Protein kinase C, mu | | | |
| TNFRSF10C | Homo sapiens TRAIL receptor 3 mRNA, | Cytokine | AF016267 | 9.08E−06 |
| | complete cds | | | |
| TERF1 | Homo sapiens telomeric repeat binding | oncogene | NM_003218 | 9.35E−06 |
| | factor (NIMA-interacting) 1 | | | |
| TGFB2 | Human transforming growth factor-beta-2 | GF | M19154 | 9.62E−06 |
| | mRNA; glioblastoma-derived T-cell | | | |
| | suppressor factor (G-TSF); bsc-1 cell | | | |
| | growth inhibitor; polyergin; cetermin | | | |
| ALDH7 | Human aldehyde dehydrogenase ALDH7 mRNA | ALDH | U10868 | 1.01E−05 |
| TTF1 | transcription termination factor, RNA polymerase | polymerase, TF | X83973 | 1.05E−05 |
| TGFBR1 | Human activin receptor-like kinase | GF, Signal | L11695 | 1.05E−05 |
| | (ALK-5) mRNA, complete cds | | | |
| ERCC3 | Human DNA repair helicase (ERCC3) | TF | M31899 | 1.11E−05 |
| | mRNA, complete cds | | | |
| CSF1R | Human macrophage colony stimulating | oncogene | X03663 | 1.18E−05 |
| | factor I receptor precursor (CSF1R); fms | | | |
| | proto-oncogene (c-fms) | | | |
| ABCB10 | Human ATP-binding cassette protein | ABC transporter | U18237 | 1.19E−05 |
| | mRNA 06B09 clone, partial cds | | | |
| STAT1 | Homo sapiens transcription factor ISGF-3 | Signal, TF | M97935 | 1.19E−05 |
| | mRNA, complete cds | | | |
| MX2 | Human interferon-induced cellular | Cytokine | M30818 | 1.22E−05 |
| | resistance mediator protein (MxB) mRNA | | | |
| SCYA1 | Human secreted protein (I-309) mRNA; | Cytokine | M57502 | 1.28E−05 |
| | Small inducible cytokine A1 (I-309, | | | |
| | homologous to mouse Tca-3) | | | |
| RBL2 | Human retinoblastoma-like protein 2 | Signal, TF | X74594 | 1.32E−05 |
| | (RBL2; RB2); 130-kDa | | | |
| | retinoblastoma-associated protein (p130) | | | |
| VCAM1 | Homo sapiens vascular cell adhesion | glucocorticoids | NM_001078 | 1.38E−05 |
| | molecule 1 (VCAM1) | (Cortisol) | | |
| MADH4 | Human homozygous deletion target in | Signal, Supressor, TF | U44378 | 1.39E−05 |
| | pancreatic carcinoma (DPC4); mothers | | | |
| | against dpp homolog 4 (SMAD4) | | | |
| ADH2 | Human class I alcohol dehydrogenase | ADH | M21692 | 1.46E−05 |
| | (ADH2) beta-1 subunit mRNA | | | |
| ISGF3G | Human IFN-responsive transcription factor | Signal, TF | M87503 | 1.48E−05 |
| | subunit mRNA; Interferon-stimulated | | | |
| | transcription factor 3, gamma (48 kD); p48 | | | |
| SCYA3 | Human macrophage inflammatory protein | Cytokine, Signal | M23452 | 1.48E−05 |
| | (G0S19-1) mRNA, Small inducible | | | |
| | cytokine subfamily A (Cys—Cys), member 3; Mip-1a | | | |
| RAB11A | Homo sapiens rab11a GTPase mRNA, complete cds. | oncogene | AF000231 | 1.50E−05 |
| ABL2 | Human tyrosine kinase arg gene mRNA | oncogene | M35296 | 1.55E−05 |
| IL6R | Human mRNA for interleukin-6 (IL-6) receptor | Cytokine, Signal | X12830 | 1.77E−05 |
| DTR | Human heparin-binding EGF-like growth factor mRNA | GF | M60278 | 1.81E−05 |
| | (HBEGF); diphtheria toxin receptor (DTR) | | | |
| ALDH9 | Human gamma-aminobutyraldehyde dehydrogenase mRNA | ALDH | U34252 | 1.85E−05 |
| SKIL | Human sno oncogene mRNA for snoN | oncogene, Signal | X15219 | 1.85E−05 |
| | protein, ski-related | | | |
| AKR1B1 | Homo sapiens aldo-keto reductase family 1, | hyperosmotic stress | NM_001628 | 1.90E−05 |
| | member B1 (aldose reductase) | | | |
| CDK2 | Human cdc2-related protein kinase mRNA, | CellCycle, Signal | M68520 | 1.92E−05 |
| | complete cds | | | |
| ABCE1 | H. sapiens mRNA for 2′-5′ oligoadenylate | ABC transporter | X74987 | 2.01E−05 |
| | Binding protein | | | |
| ST13 | Homo sapiens putative tumor suppressor | Supressor | U17714 | 2.03E−05 |
| | ST13 (ST13) mRNA, complete cds | | | |
| CFLAR | Homo sapiens Casper mRNA; CASP8 and | Appoptosis, Signal | AF010127 | 2.08E−05 |
| | FADD-like apoptosis regulator; I-FLICE | | | |
| NR5A2 | Homo sapiens hepatocytic transcription | NR5, TF | U80251 | 2.14E−05 |
| | factor (hB1F) mRNA, complete cds | | | |
| PDGFRA | Human platelet-derived growth factor receptor | GF, Signal | M21574 | 2.19E−05 |
| | alpha (PDGFRA) mRNA; CD140A antigen | | | |
| IGF2 | Human insulin-Ikegrowth factor II mRNA, | GF | J03242 | 2.21E−05 |
| | complete cds | | | |
| AADAC | Human arylacetamide deacetylase mRNA | esterase | L32179 | 2.22E−05 |
| EP300 | Human p300 protein mRNA, complete cds | Signal, TF | U01877 | 2.30E−05 |
| TPR | H. sapiens tpr mRNA; Translocated | oncogene | X66397 | 2.32E−05 |
| | promoter region (to activated MET oncogene) | | | |
| CYP3A4 | Homo sapiens cytochrome P450-3A4 | p450 | AF182273 | 2.32E−05 |
| | (CYP3A4) mRNA, complete cds | | | |
| POLR2G | polymerase (RNA) II (DNA directed) polypeptide G | polymerase | U20659 | 2.44E−05 |
| SELL | selectin L (lymphocyte adhesion molecule 1) | Selectin | M25280 | 2.45E−05 |
| HRAS | Homo sapiens v-Ha-ras Harvey rat sarcoma | oncogene, Signal | NM_005343 | 2.46E−05 |
| | viral oncogene homolog (HRAS) | | | |
| CSNK2A1 | Human casein kinase II alpha subunit | Signal | M55265 | 2.54E−05 |
| | mRNA, complete cds. | | | |
| GNG3 | Homo sapiens guanine nucleotide binding | Signal | NM_012202 | 2.54E−05 |
| | protein (G protein), gamma 3 (GNG3), mRNA | | | |
| TGFB1 | Human transforming growth factor-beta | GF, Signal | X02812 | 2.61E−05 |
| | (TGF-beta; TGFB) | | | |
| TNFRSF1A | H. sapiens TNF-R mRNA for tumor necrosis | Cytokine, Signal | X55313 | 2.62E−05 |
| | factor receptor type 1. | | | |
| ABCB1 | Homo sapiens P-glycoprotein (PGY1) mRNA (MDR1) | glucocorticoids | M14758 | 2.63E−05 |
| | | (Cortisol) | | |
| BRCA1 | Human breast and ovarian cancer | Signal, Supressor | U14680 | 2.67E−05 |
| | susceptibility (BRCA1) | | | |
| MAPK13 | Homo sapiens stress-activated protein | Stress | AF004709 | 2.82E−05 |
| | kinase 4 (SAPK4) mRNA, complete cds | | | |
| RPC62 | polymerase (RNA) III (DNA directed) (62 kD) | polymerase | U93867 | 2.87E−05 |
| SCYB5 | H. sapiens ENA-78 mRNA; Small inducible | Cytokine, Signal | X78686 | 3.10E−05 |
| | cytokine subfamily B (Cys-X-Cys), | | | |
| | member 5 (epithelial-derived | | | |
| | neutrophil-activating peptide 78) | | | |
| ATP6H | ATPase, H+ transporting, lysosomal | ATPase | Y15286 | 3.12E−05 |
| | (vacuolar proton pump) 9 kD | | | |
| THY1 | Homo sapiens Thy-1 cell surface antigen | Signal | NM_006288 | 3.13E−05 |
| | (THY 1), mRNA | | | |
| ABCB6 | Homo sapiens clone 24410 ABC transporter | ABC transporter | AF070598 | 3.26E−05 |
| | mRNA, partial cds | | | |
| STIP1 | Homo sapiens | stress | NM_006819 | 3.28E−05 |
| | stress-induced-phosphoprotein 1 | | | |
| | (Hsp70/Hsp90-organizing protein) | | | |
| IL2RB | Human interleukin 2 receptor beta chain | Cytokine, Signal | M26062 | 3.31E−05 |
| | (p70-75) mRNA, complete cds | | | |
| AP1S2 | Homo sapiens adaptor-related protein | AP-1 | NM_003916 | 3.41E−05 |
| | complex 1, sigma 2 subunit (AP1S2) | | | |
| TRA@ | Human mRNA for T-cell receptor alpha | Signal | X02592 | 3.46E−05 |
| | chain (TCR-alpha). | | | |
| EGFR | Human mRNA for precursor of epidermal | oncogene, Signal | X00588 | 3.55E−05 |
| | growth factor receptor | | | |
| CSF3 | Human mRNA for granulocyte | Cytokine, Signal | X03438 | 3.73E−05 |
| | colony-stimulating factor (G-CSF). | | | |
| GSTM3 | Human glutathione transferase M3 (GSTM3) mRNA | GSTM | J05459 | 3.74E−05 |
| CYP8B1 | Homo sapiens sterol 12-alpha hydroxylase | P450 | AF090318 | 3.80E−05 |
| | CYP8B1 (Cyp8b1) mRNA, partial cds | | | |
| TIMP3 | Human tissue inhibitor of metalloproteinase- | Signal | U02571 | 3.80E−05 |
| | 3 precursor (TIMP-3) mRNA, complete cds | | | |
| UGT2B4 | Human mRNA for liver microsomal | UGT | Y00317 | 3.83E−05 |
| | UDP-glucuronosyltransferase (UDPGT). | | | |
| PAK2 | Human p21-activated protein kinase | Signal | U24153 | 3.90E−05 |
| | (PAK-gamma; PAK2); PAK65; S6/H4 kinase | | | |
| AFG3L2 | AFG3 (ATPase family gene 3, yeast)-like 2 | ATPase | NM_006796 | 3.97E−05 |
| MST1R | H. sapiens RON mRNA for tyrosine kinase; | Signal | X70040 | 4.05E−05 |
| | Macrophage stimulating 1 receptor | | | |
| | (c-met-related tyrosine kinase) | | | |
| HSPA10 | Homo sapiens heat shock 70 kD protein 10 | hsp | NM_006597 | 4.15E−05 |
| | (HSC71) (HSPA10), mRNA | | | |
| AKAP2 | Homo sapiens A kinase (PRKA) anchor | Signal | NM_007203 | 4.44E−05 |
| | protein 2 (AKAP2) | | | |
| ABCB7 | Homo sapiens ATP binding cassette | ABC transporter | AF038950 | 4.55E−05 |
| | transporter mRNA, complete cds | | | |
| CCNC | Human cyclin mRNA | CellCycle | M74091 | 4.95E−05 |
| NPR2L | Homo sapiens candidate tumor suppressor | Supressor | AF040708 | 5.01E−05 |
| | gene 21 protein mRNA, complete cds | | | |
| JAK1 | Human protein-tyrosine kinase (JAK1) | Signal | M64174 | 5.04E−05 |
| | mRNA, Janus kinase 1 | | | |
| AKAP9 | Homo sapiens A kinase (PRKA) anchor | Signal | NM_005751 | 5.09E−05 |
| | protein (yotiao) 9 (AKAP9) | | | |
| ABCC5 | Homo sapiens SMRP mRNA, complete cds | ABC transporter | AB005659 | 5.19E−05 |
| STAC | Homo sapiens mRNA for stac, (src homology | Signal | D86640 | 5.19E−05 |
| | three (SH3) and cysteine rich domain) | | | |
| PRKDC | Homo sapiens DNA-dependent protein | Signal | U47077 | 5.70E−05 |
| | kinase catalytic subunit (DNA-PKcs) mRNA | | | |
| ABCD2 | Homo sapiens mRNA for adrenoleukodystrophy | ABC transporter | AJ000327 | 6.10E−05 |
| | related protein (ALDR). | | | |
| MAP2K1 | Homo sapiens ERK activator kinase | Signal | L11284 | 6.21E−05 |
| | (MEK1) mRNA | | | |
| RAP1A | Human ras-related protein (Krev-1) mRNA, | Supressor | M22995 | 6.33E−05 |
| | complete cds | | | |
| GNG10 | Human G protein gamma-10 subunit mRNA; | Signal | U31383 | 6.48E−05 |
| | Guanine nucleotide binding protein 10 | | | |
| MADH2 | Human mad protein homolog (hMAD-2) | Signal, TF | U68018 | 6.65E−05 |
| | mRNA; JV18-1.MADR2 OR SMAD2 | | | |
| NR3C1 | Human glucocorticoid receptor alpha | glucocorticoids | M10901 | 6.73E−05 |
| | mRNA, complete cds | (Cortisol) | | |
| RBBP1 | Homo sapiens retinoblastoma-binding | Signal | NM_002892 | 7.10E−05 |
| | protein 1 (RBBP1) mRNA | | | |
| PTPRC | Human mRNA for T200 leukocyte common | Signal | Y00062 | 7.43E−05 |
| | antigen (CD45, LC-A). | | | |
| CDC27 | Human homologue of S. pombe nuc2+ and | CellCycle | U00001 | 7.77E−05 |
| | A. nidulans bimA; Cell division cycle 27 | | | |
| HSPCA | Homo sapiens Hsp89-alpha-delta-N | hsp | AF028832 | 7.88E−05 |
| | mRNA; Heat shock 90 kD protein 1, alpha | | | |
| RAB9 | Human small GTP binding protein Rab9 | oncogene | U44103 | 9.21E−05 |
| | mRNA, complete cds. | | | |
| ING1 | Homo sapiens growth inhibitor p33ING1 | Signal, Supressor | AF001954 | 1.18E−04 |
| | (ING1) mRNA, complete cds | | | |
| KRAS2 | Human K-ras oncogene protein mRNA (KRAS2) | oncogene | M54968 | 1.31E−04 |
| RAB4 | Homo sapiens GTP-binding protein | oncogene | M28211 | 1.47E−04 |
| | (RAB4) mRNA, complete cds. | | | |
| NTF5 | Human neurotrophin-4 (NT-4) gene; | GF | M86528 | 1.99E−04 |
| | neurotrophin 5 (neurotrophin 4/5) (NTF5) | | | |
| NFRKB | Human R kappa B mRNA, complete cds | Signal | U08191 | 2.29E−04 |
| TAF2F | TATA box binding protein (TBP)-associated | polymerase, TF | U18062 | 2.79E−04 |
| | factor, RNA polymerase II, F, 55 kD | | | |
| CDC25B | Human cdc25B mRNA, complete cds. | CellCycle | M81934 | 5.63E−04 |

TABLE 2
List of genes exhibiting variable expression in non-T-cell-derived samples

| | | | GenBank | |
|---|---|---|---|---|
| Symbol | Name | Category | (Acc. No.) | p-log |
| ICAM1 | Human intercellular adhesion molecule-1 | Signal | J03132 | 1.11E−09 |
| | (ICAM-1) mRNA, CD54 | | | |
| IL18R1 | Human putative transmembrane receptor | Cytokine, Signal | U43672 | 1.14E−09 |
| | IL-1Rrp mRNA, complete cds | | | |
| CDC42 | Human GTP-binding protein (G25K) | CellCycle | M35543 | 1.49E−08 |
| | mRNA, complete cds | | | |
| SMARCA3 | SWI/SNF related, matrix associated, actin | ATPase | Z46606 | 3.95E−08 |
| | dependent regulator of chromatin, | | | |
| | subfamily a, member 3 | | | |
| RGS14 | Homo sapiens regulator of G protein | Signal | AF037195 | 5.44E−08 |
| | signaling RGS14 mRNA, complete cds. | | | |
| COX15 | Homo sapiens COX15 (yeast) homolog, | mitochondria & stress | NM_004376 | 6.43E−08 |
| | cytochrome c oxidase assembly protein | | | |
| | (COX15) | | | |
| AKAP11 | A kinase (PRKA) anchor protein 11 | Signal | AB014529 | 1.68E−07 |
| | (AKAP11); Homo sapiens mRNA for | | | |
| | KIAA0629 protein, partial cds | | | |
| RIPK2 | Homo sapiens serine/threonine kinase | Appoptosis, Signal | AF027706 | 1.88E−07 |
| | RICK (RICK) mRNA; RIP2 | | | |
| TCF17 | Homo sapiens HKL1 mRNA, complete cds | Signal, TF | D89928 | 1.92E−07 |
| CDC25B | Human cdc25B mRNA, complete cds. | CellCycle | M81934 | 2.40E−07 |
| GZMA | Human Hanukah factor serine protease | esterase | M18737 | 2.49E−07 |
| | (HuHF) mRNA (cytotoxic | | | |
| | T-lymphocyte-associated serine esterase 3) | | | |
| CHST4 | Homo sapiens carbohydrate | sulfotransferase | NM_005769 | 3.46E−07 |
| | (N-acetylglucosamine 6-O) | | | |
| | sulfotransferase 4 (CHST4) | | | |
| IL1R2 | H. sapiens IL-1R2 mRNA for type II | Cytokine | X59770 | 4.56E−07 |
| | interleukin-1 receptor, (cell line CB23). | | | |
| BCL2 | Human bcl-2 mRNA; apoptosis regulator | oncogene, Signal | M14745 | 4.81E−07 |
| | bcl2 | | | |
| ARHI | Homo sapiens putative tumor supressor | Signal, suppressor | U96750 | 4.88E−07 |
| | NOEY2 mRNA; Ras homolog gene family, | | | |
| | member I | | | |
| CR2 | Complement component (3d/Epstein Barr | Signal | M26004 | 5.88E−07 |
| | virus) receptor 2; CD21 | | | |
| RPA1 | Replication protein A1 (70 kD) | Signal | M63488 | 6.72E−07 |
| CD3Z | Human T cell receptor zeta-chain mRNA, | Signal | J04132 | 7.14E−07 |
| | complete cds | | | |
| POLR2H | Human RNA polymerase II subunit | polymerase | U37689 | 7.28E−07 |
| | (hsRPB8) mRNA; polymerase (RNA) II | | | |
| | (DNA directed) polypeptide H | | | |
| PEMT | Homo sapiens mRNA for | methytransferase | AB029821 | 9.72E−07 |
| | phosphatidylethanolamine | | | |
| | N-methyltransferase, complete cds | | | |
| E2F5 | Human transcription factor E2F-5 mRNA, | TF | U15642 | 1.00E−06 |
| | complete cds | | | |
| MAD | Homo sapiens antagonizer of myc | TF | L06895 | 1.00E−06 |
| | transcriptional activity (Mad) mRNA, | | | |
| | complete cds | | | |
| CSF1 | Human macrophage-specific | Cytokine, Signal | M37435 | 1.34E−06 |
| | colony-stimulating factor (CSF-1) | | | |
| | mRNA, complete cds | | | |
| RAB7L1 | Homo sapiens mRNA for small | oncogene | D84488 | 1.49E−06 |
| | GTP-binding protein, complete cds | | | |
| NFATC3 | Homo sapiens NF-AT4c mRNA, complete | Signal, TF | L41067 | 1.66E−06 |
| | cds | | | |
| HSPA1L | Homo sapiens HSPA1L mRNA for Heat | hsp | D85730 | 1.87E−06 |
| | shock protein 70 testis variant, complete | | | |
| | cds; Heat shock 70 kD protein-like 1 | | | |
| GR02 | Human mRNA for macrophage | Cytokine | X53799 | 1.91E−06 |
| | inflammatory protein-2alpha (MIP2alpha,; | | | |
| | GRO2 oncogene | | | |
| ARHGEF1 | Human guanine nucleotide exchange factor | Signal | U64105 | 2.01E−06 |
| | p115-RhoGEF mRNA, partial cds; Rho | | | |
| | guanine nucleotide exchange factor (GEF) 1 | | | |
| GHSR | Homo sapiens growth hormone | GH | NM_004122 | 2.14E−06 |
| | secretagogue receptor (GHSR) | | | |
| BAG4 | Homo sapiens silencer of death domains | Signal | AF111116 | 3.13E−06 |
| | (SODD) mRNA; BCL2-associated | | | |
| | athanogene 4 | | | |
| RBBP4 | Human chromatin assembly factor 1 p48 | Signal | X74262 | 3.13E−06 |
| | subunit (CAF1 p48 subunit); | | | |
| | retinoblastoma-binding protein 4 | | | |
| PRKDC | Homo sapiens DNA-dependent protein | Signal | U47077 | 3.36E−06 |
| | kinase catalytic subunit (DNA-PKcs) | | | |
| | mRNA | | | |
| RASSF1 | Homo sapiens putative tumor suppressor | Supressor | AF061836 | 3.49E−06 |
| | protein (RDA32) mRNA, complete cds | | | |
| SCYA2 | monocyte chemoattractant protein-1 | Cytokine, Signal | S71513 | 3.70E−06 |
| | [human, mRNA, 739 nt], MCP-1 | | | |
| ABCA1 | Homo sapiens mRNA for ATP-binding | ABC transporter | AJ012376 | 4.57E−06 |
| | cassette transporter-1 (ABC-1) | | | |
| TOP2A | Human DNA topoisomerase II (top2) | topoiosomerase | J04088 | 4.82E−06 |
| | mRNA, complete cds | | | |
| DAXX | Homo sapiens Fas-binding protein Daxx | Signal | AF015956 | 5.16E−06 |
| | mRNA, complete cds | | | |
| EGF | Human mRNA for kidney epidermal growth | GF, Signal | X04571 | 5.74E−06 |
| | factor (EGF) precursor; urogastrone | | | |
| GNRH1 | Human placenta mRNA for luteinizing | LH | X01059 | 5.74E−06 |
| | hormone releasing hormone precursor | | | |
| | (LHRH). | | | |
| TNFAIP6 | Tumor necrosis factor, alpha-induced | Cytokine, Signal | M31165 | 6.14E−06 |
| | protein 6 | | | |
| TNFRSF10B | Homo sapiens death receptor 5 (DR5) | Appoptosis | AF016268 | 6.95E−06 |
| | mRNA, Tumor necrosis factor receptor | | | |
| | superfamily, member 10b | | | |
| STK9 | serine/threonine kinase 9 | Gap-junciton | X89059 | 8.86E−06 |
| NPR2L | Homo sapiens candidate tumor suppressor | Supressor | AF040708 | 1.13E−05 |
| | gene 21 protein mRNA, complete cds | | | |
| ATM | Human ataxia telangiectasia (ATM) mRNA | Signal, Supressor | U33841 | 1.26E−05 |
| PPP3CB | Human calcineurin A2 mRNA; | Signal | M29551 | 1.32E−05 |
| FGF7 | Human keratinocyte growth factor mRNA; | GF | M60828 | 1.37E−05 |
| | fibroblast growth factor 7 (FGF-7) | | | |
| CD79B | Human immunoglobulin superfamily | Signal | M89957 | 1.68E−05 |
| | member B cell receptor complex cell | | | |
| | surface glycoprotein (IGB) mRNA, CD79B | | | |
| HSPA1A | Homo sapiens heat shock 70 kD protein 1 | hsp | NM_005345 | 1.74E−05 |
| | (HSPA1A), mRNA; Heat shock 70 kD | | | |
| | protein 1 | | | |
| IL2RG | Human mRNA for interleukin 2 receptor | Cytokine, Signal | D11086 | 1.90E−05 |
| | gamma chain | | | |
| E2F4 | Homo sapiens E2F transcription factor 4, | TF | NM_001950 | 1.95E−05 |
| | p107/p130-binding (E2F4) | | | |
| NR1D1 | Homo sapiens mRNA for Rev-ErbAalpha | NR1 | X72631 | 2.15E−05 |
| | protein (hRev gene). | | | |
| DTR | Human heparin-binding EGF-like growth | GF | M60278 | 2.36E−05 |
| | factor mRNA (HBEGF); diphtheria toxin | | | |
| | receptor (DTR) | | | |
| MSH2 | Human DNA mismatch repair protein | DNArepair | U04045 | 2.48E−05 |
| | MSH2 | | | |
| BCL3 | Human B-cell lymphoma 3-encoded protein | oncogene, Signal | M31732 | 2.49E−05 |
| | (bcl-3) mRNA, complete cds | | | |
| TGFB1 | Human transforming growth factor-beta | GF, Signal | X02812 | 2.52E−05 |
| | (TGF-beta; TGFB) | | | |
| ILFl | Human mRNA for transcription factor ILF | Cytokine, TF | X60787 | 2.58E−05 |
| GABPB1 | Homo sapiens GA-binding protein | mitochondria & stress | NM_005254 | 2.90E−05 |
| | transcription factor, beta subunit 1 (53 kD); | | | |
| | nuclear respiratory factor-2 | | | |
| CDK10 | Homo sapiens CDC2-related protein kinase | CellCycle | L33264 | 3.06E−05 |
| | (PISSLRE) mRNA; Cyclin-dependent | | | |
| | kinase (CDC2-like) 10 | | | |
| ADPRT | Human poly(ADP-ribose) polymerase | Signal | M18112 | 3.24E−05 |
| | mRNA (ADPRT), PARP | | | |
| CD3D | Homo sapiens CD3D antigen, delta | Signal | NM_000732 | 3.56E−05 |
| | polypeptide (TiT3 complex) (CD3D), | | | |
| | mRNA | | | |
| ATP2C1 | ATPase, Ca++-sequestering | ATPase | AF225981 | 3.59E−05 |
| STIP1 | Homo sapiens | stress | NM_006819 | 3.66E−05 |
| | stress-induced-phosphoprotein 1 | | | |
| | (Hsp70/Hsp90-organizing protein) | | | |
| AGTRL2 | Homo sapiens angiotensin receptor-like 2 | angiotensin | NM_005162 | 3.96E−05 |
| | (AGTRL2) | | | |
| ISGF3G | Human IFN-responsive transcription factor | Signal, TF | M87503 | 4.60E−05 |
| | subunit mRNA; Interferon-stimulated | | | |
| | transcription factor 3, gamma (48 kD); p48 | | | |
| RAB9 | Human small GTP binding protein Rab9 | oncogene | U44103 | 1.36E−04 |
| | mRNA, complete cds. | | | |

All publications, patents, and patent applications cited herein are incorporated herein by reference in their entirety.

### INDUSTRIAL APPLICABILITY

The present invention can be utilized for diagnosis of multiple sclerosis.

1. A method for evaluating multiple sclerosis comprising analyzing the expression levels of the gene group selected from among those shown in Table 1 or 2 using messenger RNA derived from the peripheral blood lymphocytes of a subject and evaluating the conditions of multiple sclerosis of the subject based on the results of the analysis.

2. The method according to claims 1, wherein the gene group includes those indicated by the symbols RGS14, CHST2, NR4A2, MAPK1, SMARCA3, TPST2, ATP6D, TCF17, ARHI, HSPA1A, AGTRL2, and PTPN6.

3. The method according to claim 2, wherein the gene group further includes at least one gene selected from among those indicated by the symbols CHST4, GHSR, Cox15, IL18R1, AKAP11, CDC42, HSPA1L, RAB7L1, POLR2H, GRO2, PEMT, RPA1, and NFATC3:

4. The method according to claim 3, wherein the gene group further includes at least one gene selected from among those indicated by the symbols ICAM1, CDC25B, IL1R2, CR2, CD3Z, MAD, CSF1, ARHGEF1, PRKDC, RASSF1, SCYA2, and ABCA1.

5. The method according to claim 4, wherein the gene group further includes at least one gene selected from among those indicated by the symbols RIPK2, NFKBIE, TNFAIP3, DAXX, TNFSF10, BAG1, TOP1, ADPRT, CREB1, MYC, BAG4, RBBP4, GZMA, BCL2, and E2F5.

6. The method according to claim 2, wherein the messenger RNA is derived from CD3⁺ T-cells separated from the peripheral blood lymphocyte.

7. The method according to claim 3, wherein the messenger RNA is derived from CD3⁺ T-cells separated from the peripheral blood lymphocyte.

8. The method according to claim 1, wherein the gene group includes those indicated by the symbols ICAM1, CDC25B, IL1R2, CR2, CD3Z, MAD, CSF1, ARHGEF1, PRKDC, RASSF1, SCYA2, and ABCA1.

9. The method according to claim 8, wherein the messenger RNA is derived from CD3⁻ non-T-cells separated from the peripheral blood lymphocyte.

10. The method according to claim 1, wherein the gene group includes those indicated by the symbols RIPK2, NFKBIE, TNFAIP3, DAXX, TNFSF10, BAG1, TOP1, ADPRT, CREB1, MYC, BAG4, RBBP4, GZMA, BCL2, and E2F5.

11. A chip for evaluating the condition of multiple sclerosis, which has probes that specifically bind to each gene in the gene group selected from among those shown in Table 1 or 2 immobilized on its surface.

12. The chip according to claim 11, wherein the gene group includes those indicated by the symbols RGS14, CHST2, NR4A2, MAPK1, SMARCA3, TPST2, ATP6D, TCF17, ARHI, HSPA1A, AGTRL2, and PTPN6.

13. The chip according to claim 12, wherein the gene group further includes at least one gene selected from among those indicated by symbols CHST4, GHSR, COX15, IL18R1, AKAP11, CDC42, HSPA1L, RAB7L1, POLR2H, GRO2, PEMT, RPA1, and NFATC3.

14. The chip according to claim 13, wherein the gene group further includes at least one gene selected from among those indicated by symbols ICAM1, CDC25B, IL1R2, CR2, CD3Z, MAD, CSF1, ARHGEF1, PRKDC, RASSF1, SCYA2, and ABCA1.

15. The chip according to claim 11, wherein the gene group includes those indicated by the symbols RIPK2, NFKBIE, TNFAIP3, DAXX, TNFSF10, BAG1, TOP1, ADPRT, CREB1, MYC, BAG4, RBBP4, GZMA, BCL2, and E2F5.

16. A commercial package for evaluating the conditions of multiple sclerosis, which comprises a primer or probe specific for each gene in the gene group shown in Table 1 or 2.

17. The commercial package according to claim 16, wherein the gene group includes those indicated by the symbols RGS14, CHST2, NR4A2, MAPK1, SMARCA3, TPST2, ATP6D, TCF17, ARHI, HSPA1A, AGTRL2, and PTPN6.

18. The commercial package according to claim 17, wherein the gene group includes at least one gene selected from among those indicated by the symbols CHST4, GHSR, COX15, IL18R1, AKAP11, CDC42, HSPA1L, RAB7L1, POLR2H, GRO2, PEMT, RPA1, and NFATC3.

19. The commercial package according to claim 18, wherein the gene group includes at least one gene selected from among those indicated by the symbols ICAM1, CDC25B, IL1R2, CR2, CD3Z, MAD, CSF1, ARHGEF1, PRKDC, RASSF1, SCYA2, and ABCA1.

20. The commercial package according to claim 16, wherein the gene group includes those indicated by the symbols RIPK2, NFKBIE, TNFAIP3, DAXX, TNFSF10, BAG1, TOP1, ADPRT, CREB1, MYC, BAG4, RBBP4, GZMA, BCL2, and E2F5.
